(19) United States
(12) Patent Application Publication
(10) Pub. No.: US 2026/0301951 A1
(43) Pub. Date: Oct. 1, 2026

(54) SYSTEM AND METHOD FOR ASSESSING A RISK OF BONE LOSS IN A PATIENT

(71) Applicant: THE UNITED STATES GOVERNMENT, AS REPRESENTED BY THE SECRETARY OF THE ARMY, Fort Detrick, MD (US)

(72) Inventors: Joseph W. GALVIN, Yelm, WA (US); Zachary T. COLBURN, Olympia, WA (US)

(21) Appl. No.: 19/479,602

(22) PCT Filed: Apr. 29, 2024
(86) PCT No.: PCT/US2024/026786

§ 371 Date: Oct. 28, 2025

Related U.S. Application Data
(60) Provisional application No. 63/462,611, filed on Apr. 28, 2023.

(51) Int. Cl. G16H 50/20; C12Q 1/6883; G16H 50/50

(52) U.S. Cl. CPC G16H 50/20; C12Q 1/6883; G16H 50/50; C12Q 2600/158; G01N 2800/108

(57) ABSTRACT

A method is provided for assessing a risk of bone loss in a patient. The method includes receiving, at a processor, first data that indicates measured values of a panel of biomarkers in a sample from a patient. A value of each biomarker in the panel corresponds to a level of the biomarker in the sample. The method also includes determining, with the processor, second data that indicates a prediction that the patient has bone loss above a threshold value based on the first data and a classifier model. A method is also provided for determining the classifier model. A system is further provided for performing one of the disclosed methods.

### BACKGROUND

Anterior shoulder instability is a common diagnosis in young athletes with an incidence in the US general population of 0.08 per thousand-person years (Owens et al, JBJS 2009, Waterman et al, Sports Health 2016). The outcomes of surgically-managed anterior shoulder labral tears with instability are favorable in this population (Arciero et al., AJSM 1994, DeBerardino et al., AJSM 2001, Bottoni et al, AJSM 2020, Provencher et al., AJSM 2005). However, delayed or non-operative management risks the development of chronic labral tears and attritional bone loss of the glenoid and humerus secondary to repeat instability events (Dickens et al., AJSM 2019, Wolfe et al., AJSM 2020, Shaha et al., JBJS 2016).

Much is known about the mechanical factors associated with recurrent anterior shoulder instability including the influence of glenoid and humeral bone loss, glenoid version, morphologic variations of the glenoid concavity, and anterior labral morphology; however, there is a paucity of literature examining the pathobiology of anterior shoulder instability and attritional glenoid bone loss (Giacomo et al., Arthroscopy 2014, Eichinger et al., AJSM 2016, Moroder et al., AJSM 2019, Vaswani et al., Arthroscopy 2020).

Much of the literature examining biomarkers in young patients with anterior glenohumeral instability has sought to identify protein biomarkers associated with the development of shoulder instability. Owens et al. conducted a prospective study of 1050 young athletes and found that increased serum relaxin concentration was associated with the subsequent likelihood of acute shoulder instability (Owens et al., Orthopedics 2016). Additionally, in another prospective study Owens et al. examined the relationship between baseline collagen biomarkers and development of subsequent shoulder instability. The authors found lower baseline collagen type II cleavage levels in patients who developed shoulder instability (Owens et al., Orthopedics 2017). In a study evaluating whether preoperative serum protein biomarkers of cartilage turnover and inflammation are associated with injury severity in shoulder instability patients, Yu et al. found that inflammatory biomarkers including HS-CRP, IL-8, and MIP-1b were not associated with specific shoulder lesions. However, Cartilage Oligomeric Matrix Protein (COMP) was elevated in Hill-Sachs lesions (Yu et al., Translational Sports Medicine).

### SUMMARY

The inventors of the present invention recognized that conventional methods for assessing risk of bone loss in patients have several drawbacks. For example, currently the measurement of blood or synovial biomarkers does not have a role in diagnosis or monitoring of injury severity in young patients with recurrent anterior shoulder instability. The inventors recognized that identification of a biomarker associated with severity of injury and extent of glenoid bone loss in anterior shoulder instability may provide novel information to track injury progression in patients who elect for nonoperative management, in-season athletes, and those in resource-limited environments with limited access to repeat advanced imaging studies (computed tomography (CT) scan, Magnetic Resonance Imaging (MRI)).

The inventors of the present invention recognized that it would be advantageous to identify one or more biomarkers whose levels are predictive of whether or not a patient has high risk of bone loss. The identification of serum biomarkers has the potential to not only better the understanding of this pathology, but also improve diagnosis and track injury severity and glenoid bone loss progression through a precision medicine approach.

Described herein is a model based on these particular biomarkers that can be used to predict whether a patient with an unknown level of bone loss is at risk for a high level of bone loss. Accordingly, this model provides a technological improvement in the technical area of predicting bone loss in patients since it selects particular data (e.g., selective biomarkers which are shown to be predictive indicators) for making this prediction which is not employed in conventional methods.

In a first set of embodiments, a method is provided for assessing a risk of bone loss in a patient. The method includes receiving, at a processor, first data that indicates measured values of a panel of biomarkers in a sample from a patient. The method also includes determining, with the processor, second data that indicates a prediction that the patient has bone loss above a threshold value based on the first data and a classifier model trained on values of the panel of biomarkers as input and observed bone loss above a threshold value as output.

In a second set of embodiments, a method is provided for determining a classifier model used in the method of the first set of embodiments. The method includes obtaining, on a processor, data that indicates measured values of a panel of biomarkers in a sample from a plurality of subjects. The method also includes assigning, on the processor, a result for each subject based on whether the subject has observed bone loss above a threshold value. The method also includes training a classifier model for predicting whether a patient has bone loss above the threshold value based on the measured values of the panel of biomarkers for each subject of the plurality of subjects as input and the observed bone loss of the subject as output.

In a third set of embodiments, a system is provided for performing one or both of the methods disclosed in the first and second set of embodiments. The system includes a device configured to measure values of a panel of biomarkers in a sample. The system also includes a processor and a memory including one or more sequences of instructions. The memory and the one or more sequences of instructions are configured to, with the processor, cause the system to perform the steps of one or more of the methods disclosed in the first and second set of embodiments.

### BRIEF DESCRIPTION OF THE DRAWINGS

FIG. 1 is a block diagram illustrating an example of a system for assessing risk of bone loss in a patient, according to an embodiment;

FIG. 2 is a flow chart illustrating an example of a method for assessing risk of bone loss in a patient using the system of FIG. 1, according to an embodiment;

FIG. 3 is a flow chart illustrating an example of a method for determining a classifier model used in the method of FIG. 2, according to an embodiment;

FIG. 4 is a volcano plot of the genes of Table 1 tested with tissue specimen;

FIG. 5 is a volcano plot of the genes of Table 2 tested with blood specimen;

FIG. 6A is a graph presenting the percentage of variance by each principal component and FIG. 6B is a graph presenting the cumulative percentage of variance by each principal component;

FIG. 7 is a plot presenting the coordinates of each specimen in PCA space;

FIG. 8 is a plot of lambda and binomial deviance;

FIG. 9 is a ROC curve generated by the model;

FIGS. 10A through 10E are histograms that depict a GLB biomarker gene (IFI44, IFIT1, IFIT3, OAS2 and PRKCB) expression in a blood sample;

FIG. 11 is a block diagram illustrating an example of a computer system upon which an embodiment of the invention may be implemented; and

FIG. 12 is a block diagram that illustrates a chip set upon which an embodiment of the invention may be implemented.

### DETAILED DESCRIPTION

### Introduction

There are no biomarkers or lab tests for monitoring the progression or injury severity for young patients with recurrent anterior shoulder instability and glenoid bone loss. In current medical practice, patients are required to obtain a CT scan or Magnetic Resonance Imaging (MRI) to evaluate for glenoid bone loss and monitor injury severity and progression.

The disclosed embodiments herein compare gene expression differences in the peripheral blood and tissue of young patients with recurrent anterior shoulder instability with and without significant glenoid bone loss (GBL). Additionally, a transcriptomic classifier for the reliable delineation of the severity of GBL in anterior shoulder instability patients was determined.

Accordingly, the embodiments of the present invention provide biomarker genes identified in blood specimen that code for proteins and a predictive model (also called classifier model or simply classifier) as well as a method to establish such a model, which discriminates between low and high glenoid bone loss conditions based on gene expression differences between low and high glenoid bone loss groups in specimens obtained from a patient. In addition, the method can be used to identify biomarkers in blood, lymphatic fluid, cells, tissues, and organs from the patient, and to establish a model for diagnosis of any diseases or disorders. This provides a technical advantage or technical solution to the recognized drawbacks of conventional methods in the technical area of predicting bone loss (e.g. glenoid bone loss) in patients.

By using the embodiments of the invention, the need for advanced imaging would be potentially avoided, which would reduce radiation to the patient in the case of a CT scan and the time and cost necessary for an expensive MRI.

The embodiments disclosed herein compare gene expression differences in the peripheral blood and tissue of young patients with recurrent anterior shoulder instability with and without significant glenoid bone loss (GBL). Additionally, it was sought to determine a transcriptomic classifier for the reliable delineation of the severity of GBL in anterior shoulder instability patients.

In a method disclosed herein for determining a classifier model, consecutive patients with symptomatic unidirectional anterior shoulder instability undergoing arthroscopic and open shoulder stabilization at a single institution were prospectively enrolled. Seventeen patients were included with a mean age of 26 years (range, 20-41). Seven patients had <10% GBL with a mean 2.3% (range, 0-8) and 10 patients had ≥10% GBL with mean 16.4% (range, 10-25). Blood specimens and shoulder capsular specimens obtained at the time of surgery were compared between patients with significant GBL (≥10%) N=10 and without (<10% GBL) N=7. RNA was extracted and a customized 277-gene expression panel (nCounter) was utilized. Gene expression levels were quantified using an nCounter. Differential expression analysis was performed to identify genes expressed at different levels between patients with and without significant GBL. The expression levels of the subset of genes identified were used to generate a ridge regression model to predict the degree of GBL (<10% or ≥10%).

In the method for determining a classifier model disclosed herein, nine genes were identified as significantly differentially expressed in the peripheral blood, and five of these IFIT1, IFIT3, IFI44, PRKCB, OAS2 (P values of 1×10⁻⁵, 1×10⁻⁴, 1×10⁻⁴, 1×10⁻⁴, and 6×10⁻⁴) were confirmed using non-parametric tests. No genes in the shoulder capsular specimens were differentially expressed. A model was developed using the 5 genes to accurately predict the severity of GBL. The predictive model had an accuracy of 88% (95% Cl of 64% to 99%), a sensitivity of 0.90, a specificity of 0.86, and an area under the receiver operating characteristic (AUROC) curve of 0.99 (95% Cl of 95% to 100%). Note that any model can be trained to fit the data, including a purely statistical model such as a regression model with coefficients for each input parameter or a neural network with scale and bias values for each node in each of one or more hidden layers between input and output. Alternatively, a process oriented biological cell model or biological tissue model with uncertain parameter values can be trained to set the parameter values. For purposes of illustration a regression model with coefficients for each input parameter is described in detail in the following.

There are significant gene expression differences in the peripheral blood of anterior shoulder instability patients with and without significant (?10%) GBL. This novel transcriptomic data may lead to a relevant biomarker to track glenoid bone loss and injury severity and progression in young patients with recurrent anterior shoulder instability.

### Definitions

The following terms as used herein have the following definitions. Unless otherwise defined, all technical and scientific terms used herein are intended to have the same meaning as commonly understood in the art to which this invention pertains and at the time of its filing. Although various methods and materials similar or equivalent to those described herein can be used in the practice or testing of the present invention, suitable methods and materials are described below. However, one skilled in the art should understand that the methods and materials used and described are examples and may not be the only ones suitable for use in the invention. Moreover, because measurements are subject to inherent variability, any temperature, weight, volume, time interval, pH, salinity, molarity or molality, range, concentration and any other measurements, quantities or numerical expressions given herein are intended to be approximate and not exact or critical figures, unless expressly stated to the contrary. Hence, where appropriate to the invention and as understood by those of skill in the art, it is proper to describe the various aspects of the invention using approximate or relative terms and terms of degree commonly employed in patent applications, such as: so dimensioned, about, approximately, substantially, essentially, consisting essentially of, comprising, and effective amount. The terms front, back and side are only used as a frame of reference for describing components herein and are not to be limiting in any way.

The terms “a” and “an” do not denote a limitation of quantity, but rather denote the presence of at least one of the referenced item. The modifier “about” used in connection with a quantity is inclusive of the stated value and has the meaning dictated by the context. All ranges disclosed within this specification are inclusive and are independently combinable. Furthermore, to the extent that the terms “including,” “includes,” “having,” “has,” “with,” or variants thereof are used in either the detailed description and/or the claims, such terms are intended to be inclusive in a manner similar to the term “comprising.” The terms front, back and side are only used as a frame of reference for describing components herein and are not to be limiting in any way.

The term “expression product(s)” as used herein refers to an RNA transcript of a gene or a protein encoding by the RNA transcript.

As used herein, the term “transcriptomics” refers to the study of transcriptome—the complete set of RNA, also known as expression profiling. A transcriptome captures a snapshot of the total transcripts present in a cell or tissue. Transcriptomics measures the gene expressions in different tissues or conditions, or at different times, gives information on the regulations of gene expression and allows quantifying gene expression changes. All transcriptomic techniques have been particularly useful in identifying the functions of genes and identifying those responsible for particular phenotypes. This technology can be applied to diagnostics and disease profiling, identification of novel virulence factors and host-pathogen immune interactions, identification of genes responding to environmental stresses and genes responsible for drug resistance, etc. For this technology, there are two key contemporary techniques in the field: microarrays, which quantify a set of predetermined sequences, and RNA-Seq using random primer and DNA sequencing method to record all transcripts.

Briefly, mRNA is extracted from the sample and reverse transcriptase is used to copy the mRNA into stable ds-cDNA. In microarrays, the ds-cDNA is fragmented and fluorescently labelled. The labelled fragments hybridize with an ordered array of complementary oligonucleotides, known as “probes”, arranged on a glass slide. Transcript abundance is determined by the hybridization of fluorescently labelled transcripts to these probes. The fluorescence intensity at each probe location on the array indicates the transcript abundance for that probe sequence. Groups of probes designed to measure the same transcript (i.e., hybridizing a specific transcript in different positions) are usually referred to as “probe-sets”. Microarrays require some genomic knowledge from the organism of interest, for example, in the form of an annotated genome sequence, or a library of expressed sequence tags (ESTs) that can be used to generate the probes for the array.

RNA-Seq also requires synthesis of cDNA from isolated mRNA by reverse transcriptase. The basic steps of an RNA-Seq method involves RNA extraction, RNA fragmentation, cDNA synthesis with a random primer, cDNA library amplification, end-repair, phosphorylation and poly-A tailing, adapter ligation (for binding of the cDNA fragments onto the flow cell and for providing sequencing primer binding sites), PCR amplification and sequencing to get strings of continuous sequence data in “reads”, aligned to a reference, and quantified for downstream analyses such as differential expression and alternative splicing.

As used herein, the term “nCounter gene expression analysis” refers to a method for detecting the expression of up to 800 genes in a single reaction with high sensitivity and linearity across a broad range of expression levels. The method is based on the principal idea of genome-wide (microarrays) and targeted (real-time quantitative PCR) expression profiling. The nCounter assay is a method for direct digital detection of target mRNAs of interest without the conversion of the mRNA to cDNA by reverse transcription or the amplification of the resulting cDNA by PCR. In an embodiment, the device 102 of the system 100 disclosed herein is configured to measure a value of biomarker levels in a sample based on these techniques.

Each target mRNA of interest is detected using a pair of reporter- and capture probes carrying 35- to 50-base target-specific sequence fragments. Each reporter probe carries a unique color code composed of 6 fluorophores of 4 colors at the 5′ end of the target-specific sequence fragments, which is the molecular barcode of the target gene of interest. The capture probes all carry a biotin label at the 3′ end of the target-specific sequence fragments that provides a molecular handle for attachment of target genes onto the nCounter cartridge to facilitate downstream digital detection. There is no gap on the target gene of interest between the probe hybridization sites of the reporter probe and the capture probe.

After hybridization between target mRNA and reporter-capture probe pairs in the hybridization solution, excess non-binding probes are removed and the probe/target complexes are aligned and immobilized in the nCounter cartridge, which is then placed in a digital analyzer for image acquisition and data processing. Hundreds of thousands of color codes specific for each mRNA target of interest are directly imaged on the surface of the cartridge. The expression level of a gene is measured by counting the number of times the color-coded barcode for that gene is detected, and the barcode counts are then summarized in a table (count data).

As used herein, the term “differential gene expression analysis” refers to testing and analysis methods to determine which genes are expressed at different levels between conditions. The read-count data used for differential expression analysis represents the number of sequence reads that originated from a particular gene. The higher the number of counts, the more reads associated with that gene, and the assumption that there was a higher level of expression of that gene in the sample. First, the read-count data needs to be normalized to account for differences in library sizes and RNA composition between samples. Then, the normalized counts are used to make some plots for QC at the gene and sample level. Finally, the analysis of differentially expressed genes is performed using an analysis tool such as Principal Component Analysis (PCA).

As used herein, the term “normalization” refers to the process of adjusting the raw read-count value by comparing it with a control such as a housekeeping gene, GAPDH in order to make accurate comparisons of gene expression between samples. The counts of mapped reads for each gene are proportional to the expression of RNA (“interesting”) in addition to many other factors (“uninteresting”). Normalization is the process of scaling raw count values to account for the “uninteresting” factors including sequencing depth, gene length, RNA composition, etc. In this way the expression levels are more comparable between and within samples.

As used herein “machine learning” refers to algorithms that give a computer the ability to learn without being explicitly programmed including algorithms that learn from and make predictions about data. Machine learning algorithms include, but are not limited to, decision tree learning, artificial neural networks (ANN) (also referred to herein as a “neural net”), deep learning neural network, support vector machines, rule base machine learning, random forest, logistic regression, pattern recognition algorithms, etc. The machine learning process has the ability to continually learn and adjust the classifier model as new data becomes available and does not rely on explicit or rules-based programming. Statistical modeling relies on finding relationships between variables (e.g., mathematical equations) to predict an outcome.

As used herein, the term “ROC (receiver operating characteristic curve)” refers to a graph showing the performance of a classification model at all classification thresholds. ROC curve plots two parameters: Sensitivity (true positive rate (TPR)) and 1-Specificity (false positive rate (FPR)). A perfect diagnostic test would be one with no false negative (sensitivity=1) or no false positive (specificity=1) results and would be represented by a line that started at the origin and went up the y-axis to a sensitivity=1, and then across to a false positive rate of 0 (1-specificity=1). A test that produces false positive results at the same rate as true positive results would produce an ROC on the diagonal line y=x. Any reasonable diagnostic test will display an ROC curve in the upper left triangle.

As used herein, the term “biomarker” refers to a biological molecule found in blood, other body fluids, or tissues that is measured as an indicator of normal biological processes, pathogenic processes, or responses to an exposure or intervention, including therapeutic interventions. Molecular, histologic, radiographic, or physiologic characteristics are types of biomarkers. A biomarker may be used to see how well the body responds to a treatment for a disease or condition, but it is not an assessment of how an individual feels, functions, or survives. BEST (Biomarkers, EndpointS and other Tools, a glossary issued by the FDA) defines seven biomarker categories: susceptibility/risk, diagnostic, monitoring, prognostic, predictive, pharmacodynamic/response, and safety.

As used herein, the term “multiplex qPCR” refers to a multiple real-time qPCR in one tube/well to amplify more than two nucleic acid sequences in a quantitative manner in one reaction solution. Quantitative polymerase chain reaction (qPCR) is a routinely used method for the detection and quantitation of gene expression in real time. In multiplex qPCR, two or more target genes are amplified in the same reaction, using the same reagent mix, including more than one pair of primers in the reaction for each target. This technique requires two or more probes that can be distinguished from each other and detected simultaneously. Each probe is labeled with a unique fluorescent dye, resulting in different observed colors for each assay. Usually, qPCR probe is a small DNA or RNA sequence recognizing complementary sequences of target RNA or DNA and labeled with a fluorescence reporter molecule. In an embodiment, the device 102 of the system 100 disclosed herein is configured to measure a level of biomarkers according to one or more of these techniques.

### Overview

The invention is described herein with reference to specific embodiments thereof.

Various modifications and changes, however, can be made to the invention without departing from the broader spirit and scope of the invention. The specification and drawings are, accordingly, illustrative rather than restrictive. Throughout this specification and the claims, unless the context requires otherwise, the word “comprise” and its cognates, such as “comprises” and “comprising,” imply the inclusion of a stated item, element or step or group of items, elements or steps but not the exclusion of any other item, element or step or group of items, elements or steps.

Notwithstanding that the numerical ranges and parameters setting forth the broad scope are approximations, the numerical values set forth in specific non-limiting examples are reported as precisely as possible. Any numerical value, however, inherently contains certain errors necessarily resulting from the standard deviation found in their respective testing measurements. Unless otherwise clear from the context, a numerical value presented herein has an implied precision given by the least significant digit. Thus, a value 1.1 implies a value from 1.05 to 1.15. The term “about” is used to indicate a broader range centered on the given value, and unless otherwise clear from the context implies a broader range around the least significant digit, such as “about 1.1” implies a range from 1.0 to 1.2. If the least significant digit is unclear, then the term “about” implies a factor of two, e.g., “about X” implies a value in the range from 0.5× to 2×, for example, about 100 implies a value in a range from 50 to 200. Moreover, all ranges disclosed herein are to be understood to encompass any and all sub-ranges subsumed therein. For example, a range of “less than 10” can include any and all sub-ranges between (and including) the minimum value of zero and the maximum value of 10, that is, any and all sub-ranges having a minimum value of equal to or greater than zero and a maximum value of equal to or less than 10, e.g., 1 to 4.

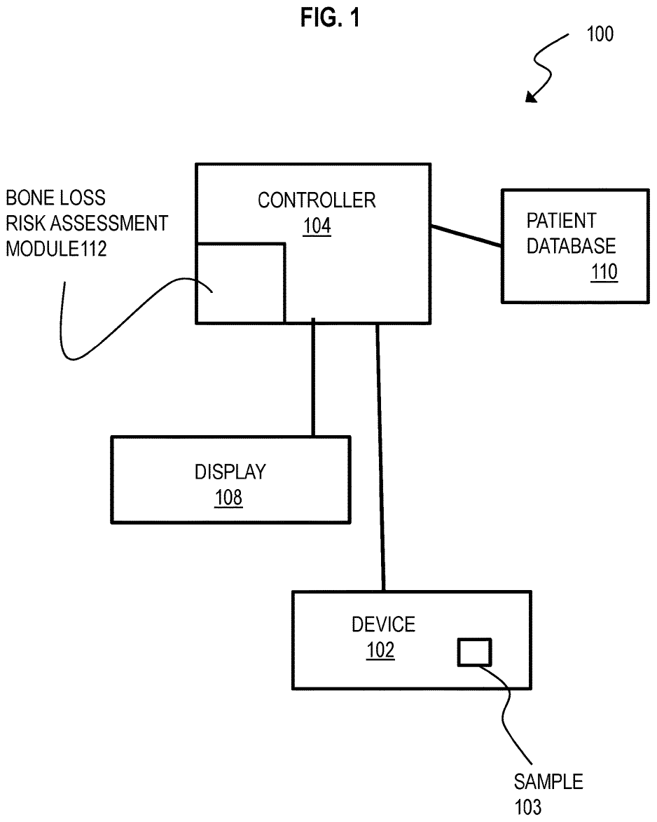

FIG. 1 is a block diagram illustrating an example of a system 100 for assessing risk of bone loss (e.g. glenoid bone loss) in a patient, according to an embodiment. In an embodiment, the system 100 includes a device 102 configured to measure values of biomarker levels in a panel obtained from a sample 103 (e.g., tissue, blood, etc.). The device 102 is communicatively coupled with a controller 104 and transmits data indicating the measured levels of the biomarker levels to the controller 104.

As further shown in FIG. 1, the system 100 includes a display 108 that is communicatively coupled with the controller 104 and is configured to output data based on a signal received from the controller 104. As further shown in FIG. 1, in some embodiments the system 100 includes a database 110 communicatively coupled with the controller 104 and configured to store data pertaining to the methods disclosed herein (e.g. a prediction that a certain patient has high bone loss along with an identifier for the patient, etc.).

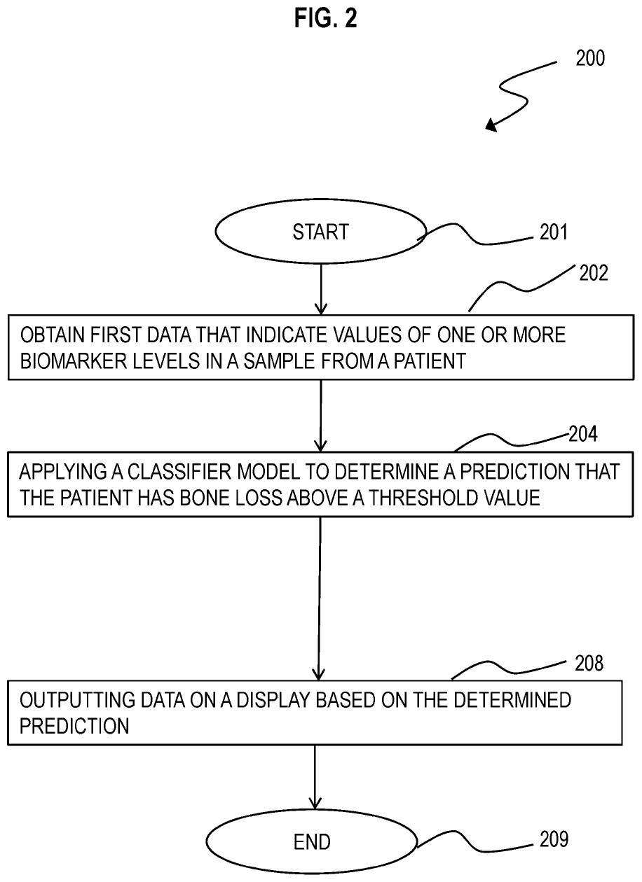

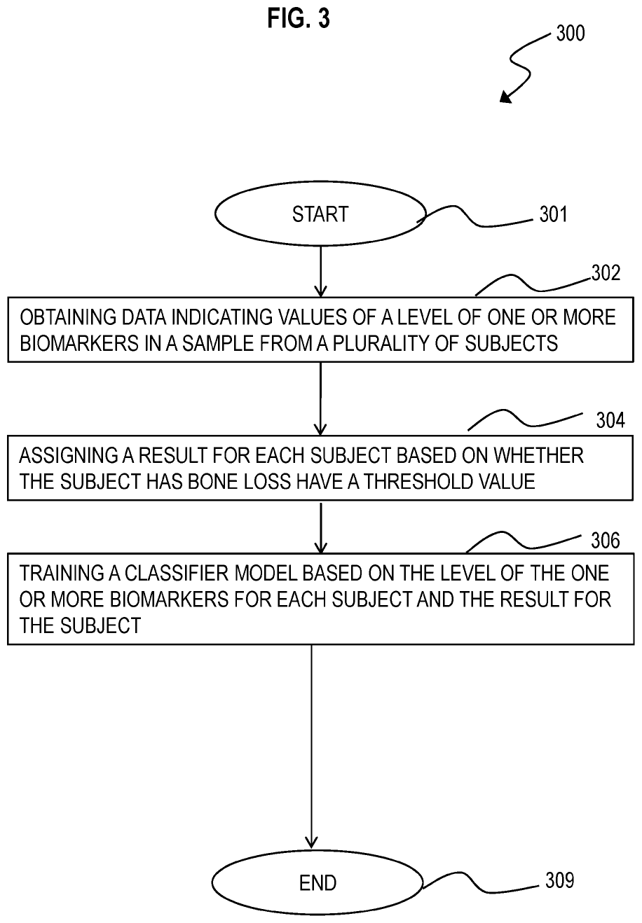

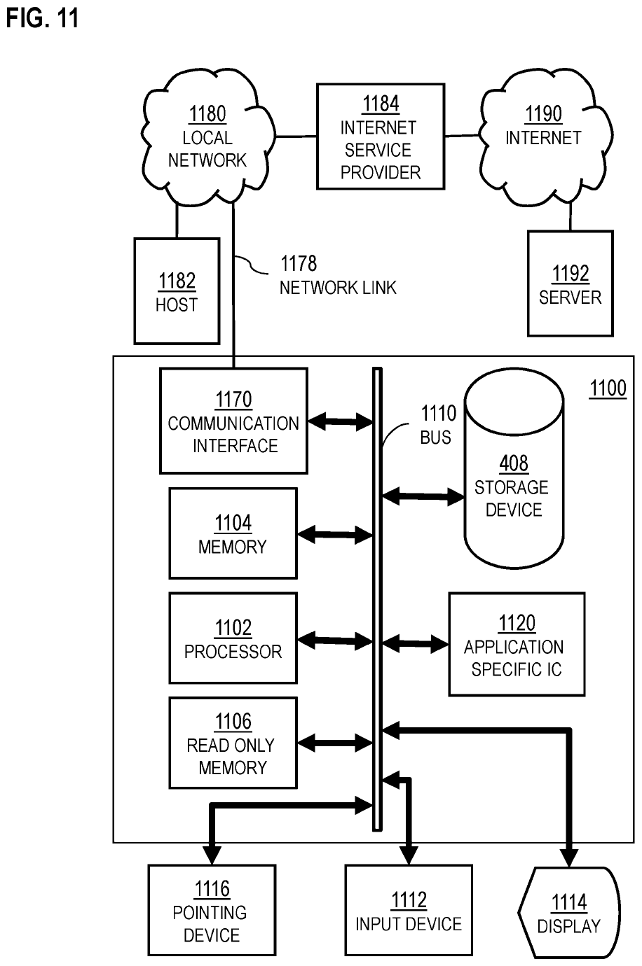

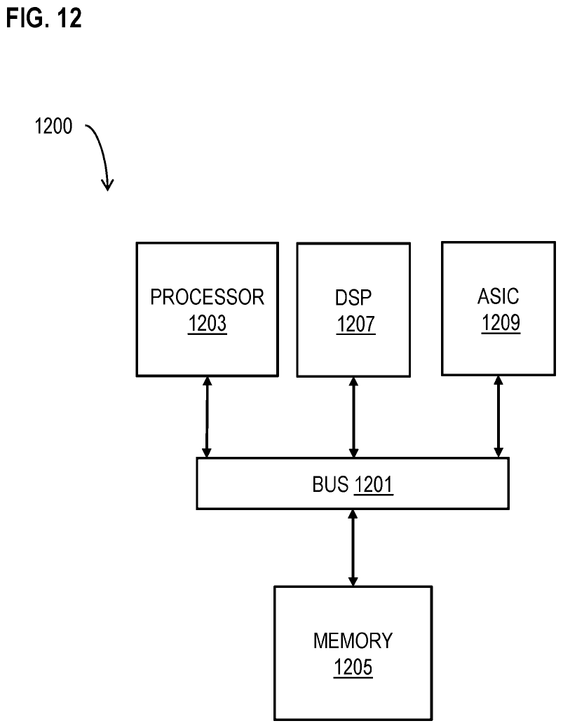

The controller 104 includes a process 112 to predict whether a patient from which the sample 103 was obtained has high bone loss (e.g. bone loss above a threshold value, such as 10%). In some embodiments, the controller 104 is a computer system 1100 as described below with reference to FIG. 11 or a chip set 1200 described below with reference to FIG. 12. The process 112 is configured to cause the controller 104 to perform one or more steps of the method 200 of FIG. 2 and/or one or more steps of the method 300 of FIG. 3. The process 112 is configured to cause the system 100 to apply coefficients to the values of the one or more biomarker levels and to determine second data that indicates a prediction that the patient has high bone loss based on applying the coefficients to the values of the one or more biomarker levels. In one embodiment, the process 112 causes the system 100 to output data on the display 108 that indicates the prediction, indicates a risk category based on the prediction and/or indicates a recommended treatment protocol based on the prediction. The hardware used to form the controller 104 of the system 100 is described in more detail below in the Hardware Section.

A method of using the system 100 to predict whether a patient has high bone loss will now be discussed. FIG. 2 is a flow chart illustrating an example of a method 200 for assessing risk of bone loss in a patient using the system 100 of FIG. 1, according to an embodiment. Although the flow diagram of FIG. 2, and subsequent flow diagram FIG. 3, is each depicted as integral steps in a particular order for purposes of illustration, in other embodiments one or more steps, or portions thereof, are performed in a different order, or overlapping in time, in series or in parallel, or are deleted, or one or more other steps are added, or the method is changed in some combination of ways.

The method 200 starts at block 201 and moves to step 202, where first data is obtained at the controller 104 that indicates values of one or more biomarker levels in the sample 103 from the patient. In one embodiment, in step 202 the first data is obtained at the controller 104 from the device 102 which measures the values of the one or more biomarker levels in the sample 103.

In an embodiment, in step 204 a trained classifier model is applied to determine a prediction that the patient is likely to have some bone loss above a threshold value. For example, in a trained regression model, coefficients are applied to each of the values of the one or more biomarker levels obtained in step 202. The classifier model is trained using the method 300 of FIG. 3 discussed below. In one embodiment, the coefficients are stored in a memory of the controller 104 The determination in step 204 is based on the value of the one or more biomarker levels obtained in step 202 and the classifier model applied in step 204. In an example embodiment using a regression model, the prediction determined in step 204 is based on an equation:

P=C₁X₁+C₂X₂+ . . . (1)

Where P is the value of the prediction determined in step 204; C₁, C₂, etc. are the coefficient values for each respective biomarker level and X₁, X₂, etc are the values of the biomarker levels from step 202. The values C₁, C₂, etc. of the coefficients are determined using the method 300 of FIG. 3.

In an embodiment, in step 208 data is output on the display 108 based on the prediction determined in step 204. In one embodiment, in step 208 the value of the prediction is output on the display 108 and/or a risk assessment of the patient based on the predicted value. In an example embodiment, a threshold value of the prediction is used to assess a risk level of whether the patient has high bone loss. In this example embodiment, the prediction threshold value is in a range from about 0.4 to about 0.6. In still other embodiments, in step 208 a treatment protocol is output on the display 108 based on the determined prediction in step 204. The method 200 then ends at block 209.

A method for training the model that is used in predicting whether a patient has high bone loss will now be discussed. In one embodiment, this method is used to determine the values of coefficients used in step 204 of the method 200. FIG. 3 is a flow chart illustrating an example of a method 300 for determining a classifier model for assessing the risk of bone loss in the patient in the method 200 of FIG. 2, according to an embodiment.

The method 300 begins at block 301 and moves to step 302 where data is obtained indicating values of one or more biomarker levels in a sample obtained from each of a plurality of subjects. In an embodiment, the device 102 of the system 100 of FIG. 1 is used to measure the values of the one or more biomarker levels in a sample 103 obtained from each of the plurality of subjects. The data is then communicated from the device 102 to the controller 104. In other embodiments, the data obtained in step 302 is not measured by the device 102 but is instead downloaded to the controller 104 from an external source (e.g., remote server).

In step 304, an observed result is assigned to each subject based on whether the subject has bone loss above the threshold value, e.g., based on a CT or MRI scan. In this embodiment, each subject used in step 302 has a known value of bone loss and thus is categorized into two categories: a first category where the bone loss is above the threshold value and a second category where the bone loss is below the threshold value. In an example embodiment, the threshold value is about 10%. In other embodiment, other thresholds are used, such as 5% or 20%. In step 304, binary values are assigned to each subject (e.g. a value of 1 is assigned to each subject who has bone loss above the threshold value and a value of 0 is assigned to each subject who has bone loss below the threshold value).

In step 306, the data obtained in step 302 (e.g., values of the one or more biomarker levels in each subject) is used to train a classifier model. For example, a regression curve is fitted to the observed result assigned in step 304 (e.g. 1 for each subject with bone loss above the threshold value and 0 for each subject with bone loss below the threshold value). In an example embodiment, the fitting step is performed using any fitting technique appreciated by one of ordinary skill in the art (e.g. least squares, principal component analysis, ridge regression, etc.). For example, the coefficients (e.g. C₁, C₂, etc. employed in step 204 of the method 200) for each biomarker level are determined based on the training step 304. These coefficient values are then stored in the memory of the controller 104 and used during the method 200 when assessing a risk of bone loss in a patient.

### Example Embodiments

The inventors of the present invention first determined what type of sample (e.g., tissue, blood, etc.) would most effectively predict whether or not a patient has high bone loss. In an embodiment, the method 300 was first performed using tissue samples, in order to determine whether an effective predictive model could be obtained using tissue samples from the plurality of subjects. Differential expression analysis was performed on tissue for the anterior group. In step 304, the specimens were split into 2 subgroups: glenoid bone loss (GBL) less than 10 or GBL greater than or equal to 10. In step 302, RNA was extracted from the specimens and subjected to nCounter analysis to obtain the result table. In step 306, differential expression results were obtained for different genes. The first 10 rows (sorted by q value) are depicted below (Table 1). As appreciated by one of ordinary skill in the art, the q value for each biomarker level indicates a difference in the level of the biomarker between the 2 subgroups and thus indicates whether the biomarker level can be used to effectively predict whether the patient has a risk of high bone loss (e.g. when the q value is below a threshold level, such as 0.05).

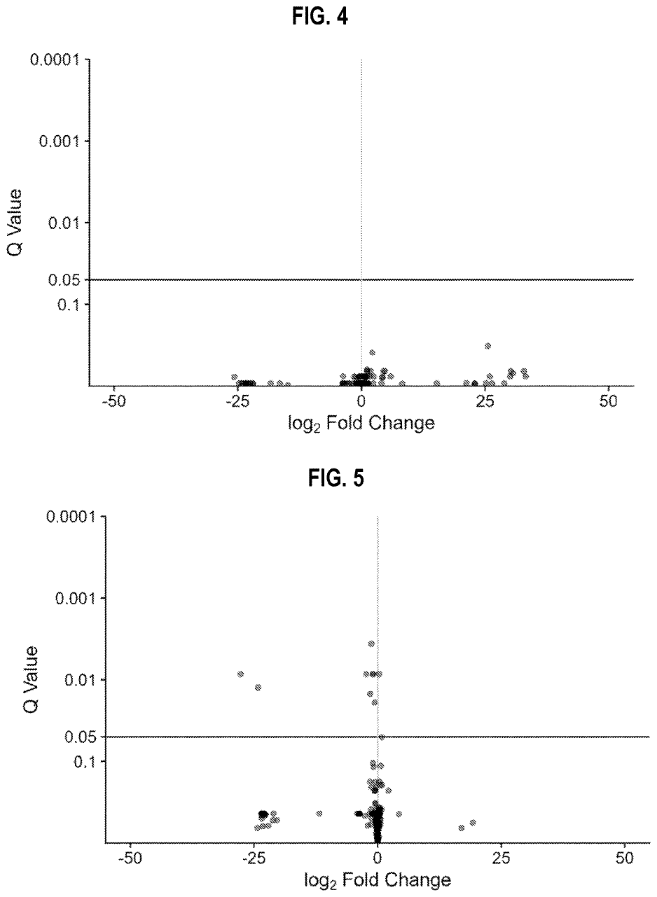

No genes were identified as differentially expressed between tissue samples of the 2 subgroups (e.g., since the q value is above the threshold value, such as about 0.05). Thus, the inventors of the present invention recognized that biomarker levels measured in step 302 from tissue samples would not be useful in determining the classifier model in 306 which is then used in the method 300. A volcano plot of Table 1 is shown in FIG. 4 which indicates that the q value of each biomarker level within both subgroups are above the threshold level (e.g., about 0.05) and thus the biomarker levels measured in the tissue samples would not be an effective parameter to predict whether or not a patient has high bone loss in the method 200.

TABLE 1
Differential Analysis of Anterior capsular tissue
specimens of patients with and without significant GBL.

| Gene | logFC | Ir | pvalue | qvalue |
|---|---|---|---|---|
| CXCL9 | 25.58264 | 10.50844 | 0.001188 | 0.32203 |
| CXCL6 | 2.212491 | 8.886926 | 0.002872 | 0.389183 |
| FGF7 | 1.178036 | 7.294704 | 0.006916 | 0.624729 |
| CCL21 | 4.426697 | 6.057549 | 0.013847 | 0.658063 |
| CCL22 | 32.86715 | 5.798991 | 0.016035 | 0.658063 |
| CCL3 | 1.840546 | 5.531679 | 0.018675 | 0.658063 |
| CCL5 | 1.184734 | 5.074323 | 0.024283 | 0.658063 |
| CCL8 | 1.158532 | 5.292561 | 0.021417 | 0.658063 |
| CCR7 | 4.775283 | 5.186386 | 0.022765 | 0.658063 |
| PTGS2 | 30.21838 | 5.128187 | 0.02354 | 0.658063 |

Where log FC indicates log base 2 fold change, Ir indicates a likelihood ratio, the pvalue is a P value without any multiple testing correction and the qvalue is a false discovery rate (p value adjusted to account for multiple comparisons).

After determining that biomarker levels in tissue samples are not effective predictors of a risk of high bone loss, the inventors of the present invention assessed whether biomarker levels in blood samples are effective predictors of high bone loss. The method 300 was repeated with blood samples. Differential expression analysis was performed on blood for the anterior group. The specimens were split into 2 subgroups: glenoid bone loss (GBL) less than 10 or GBL greater than or equal to 10. The genes that were differentially expressed (sorted by q value) are depicted below (Table 2).

As shown in Table 2 below, nine genes were identified as differentially expressed (e.g. where the q value is <0.05). A volcano plot of Table 2 is shown in FIG. 5 where the genes whose q value is <0.05 (good predictors of high bone loss) are clearly differentiated from those whose q value is >0.05 (not good predictors of high bone loss). This peripheral blood lab test provides a genomic classifier (using 5 differentially expressed genes) which can identify patients with anterior shoulder instability with at least or less than 10% glenoid bone loss, respectively.

TABLE 2
Differential Analysis of Peripheral blood specimens
of patients with and without significant GBL

| Gene | logFC | Ir | pvalue | qvalue |
|---|---|---|---|---|
| IFIT1 | 1.23899 | 18.94743 | 1.34E−05 | 0.003641 |
| CCL3 | 2.32292 | 14.63618 | 0.00013 | 0.008528 |
| FGFR2 | 27.6706 | 14.17436 | 0.000167 | 0.008528 |
| IFI44 | 0.95427 | 14.94123 | 0.000111 | 0.008528 |
| IFIT3 | 0.85664 | 14.99025 | 0.000108 | 0.008528 |
| PRKCB | 0.28161 | 13.93934 | 0.000189 | 0.008528 |
| CXCL10 | −24.167 | 12.9334 | 0.000323 | 0.012496 |
| NOD1 | 1.51282 | 12.35373 | 0.00044 | 0.014909 |
| OAS2 | 0.59126 | 11.6818 | 0.000631 | 0.019004 |

The differentially expressed genes identified above were evaluated using a non-parametric approach as well. The inventors recognized that this helps ensure that the assumptions of the statistical methods used in the above analysis do not unduly influence the results. Specifically, Wilcoxon rank sum tests were performed. The key results (the p values) are presented below (Table 3). Several genes (highlighted in yellow) did not show a significant difference using this approach. Four genes (NOD1, CCL3, CXCL10, and FGFR2) having non-significant p values were excluded from further analysis. Thus, the remaining five genes listed below are effective predictors of a risk of high bone loss.

TABLE 3
Non-parametric test confirmation for Differential
Analysis of Peripheral blood specimens

| Gene | P Value Wilcoxon Rank Sum Test |
|---|---|
| IFIT1 | 0.003 |
| OAS2 | 0.005 |
| PRKCB | 0.005 |
| IFIT3 | 0.010 |
| IFI44 | 0.014 |
| NOD1 | 0.055 |
| CCL3 | 0.070 |
| CXCL10 | 0.315 |
| FGFR2 | 0.315 |

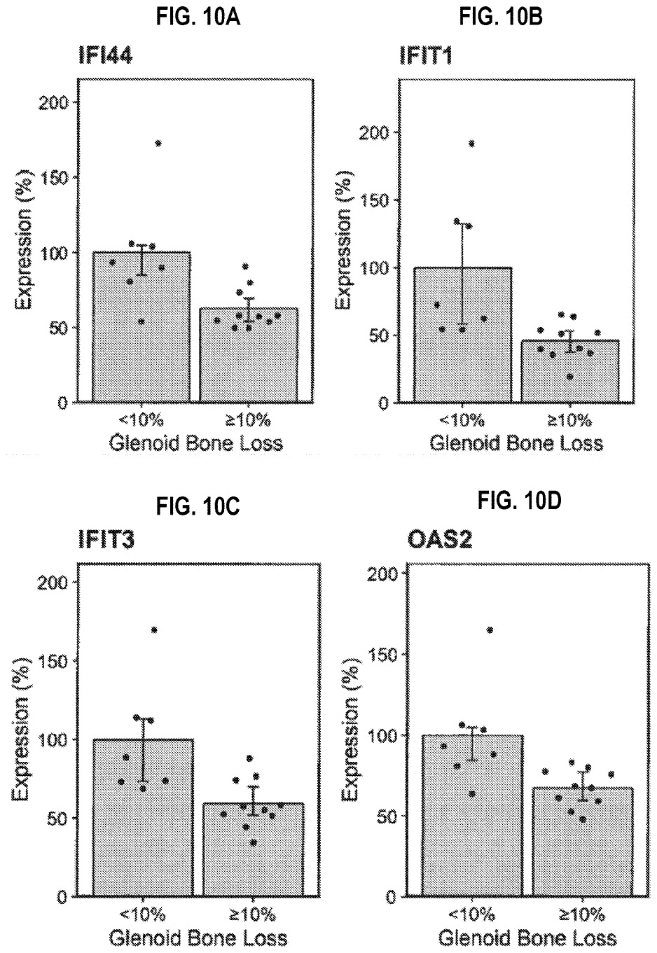

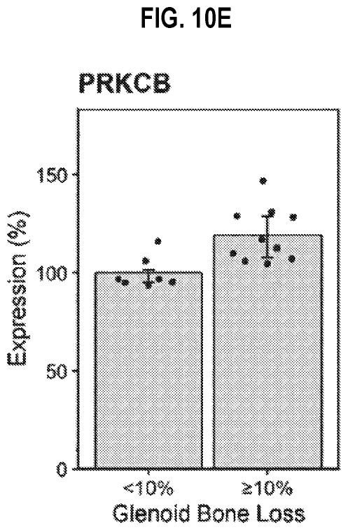

In an embodiment, in step 306 of the method 300, normalization of the gene expression count data was performed. Briefly, normalization was performed by dividing the transcript counts for each gene by the geometric mean of the transcript counts for the housekeeping genes. Principal Components Analysis (PCA) was performed on the normalized gene expression data for the top 5 genes in Table 3 (the non-highlighted genes: IFIT1, IFIT3, IFI44, PRKCB, and OAS2). The percentage of variance explained by each principal component is reproduced in Table 4. Additionally, FIGS. 10A through 10E indicate the different in the biomarker levels of each of these five genes between the 2 subgroups (bone loss <10% and <10%). FIGS. 10A through 10E confirm that the biomarker levels of these five genes can be effectively used in the method 200 of FIG. 2 to predict whether a patient has high bone loss and in the method 300 of FIG. 3 to determine an effective model (e.g., coefficient values for each biomarker level) that can be used in the method 200 of FIG. 2.

TABLE 4
Blood-PCA

| | Percentage of | Cumulative percentage |
|---|---|---|
| PC | variance | of variance |
| 1 | 76.4 | 76.4 |
| 2 | 19.0 | 95.4 |
| 3 | 3.5 | 98.9 |
| 4 | 0.6 | 99.6 |
| 5 | 0.4 | 100 |

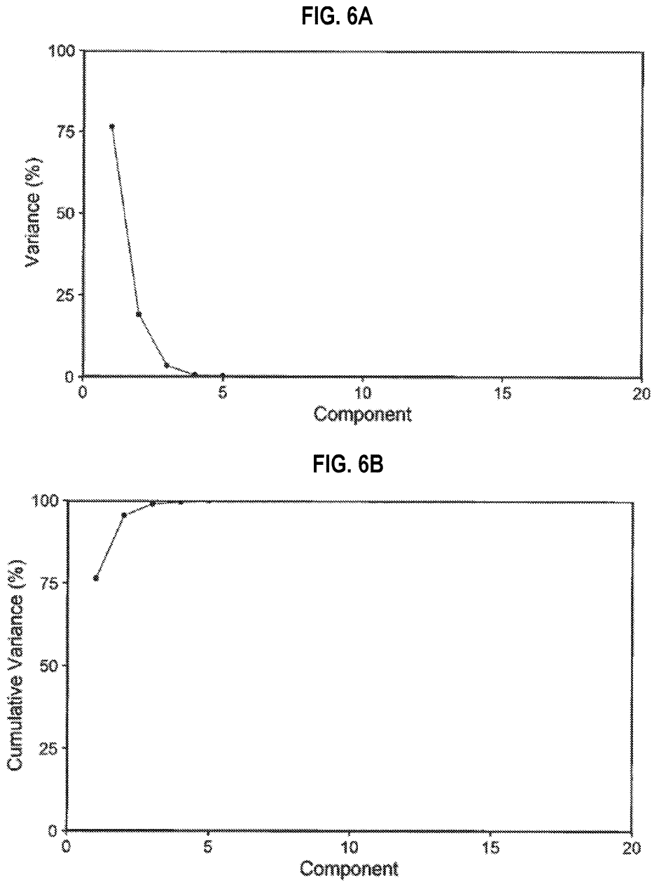

These results were plotted in FIGS. 6A and 6B, showing that PC1 and PC2 are the main components in the data set. Thus, in one embodiment, both PC1 and PC2 can be effectively used to predict whether a patient has high bone loss (method 200 of FIG. 2) and to determine a model (coefficient values in the method 300 of FIG. 3) that can be used to predict whether a patient has high bone loss.

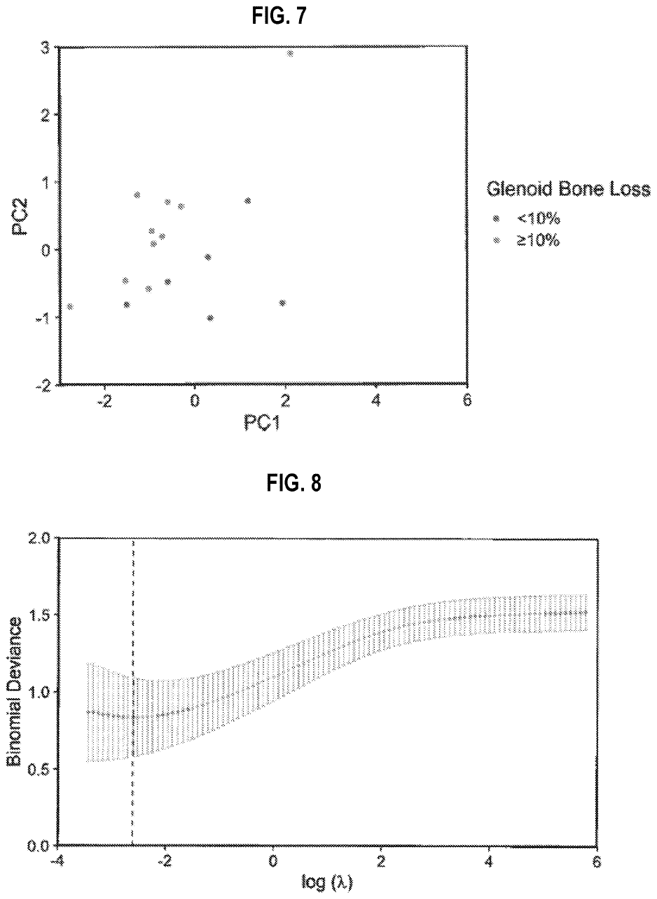

In an example embodiment, in step 306, FIG. 7 depicts the coordinates of each specimen plotted in PCA space to present the subjects of GBL<10% and GBL≥10% in PC1 and PC2 2D space. In this embodiment, in step 306 the data for PC1 and PC2 obtained in step 302 is fit with the result data from step 304 (e.g. 1 for each subject with bone loss >10% or the red dots in FIG. 7 and 0 for each subject with bone loss <10% or the blue dots in FIG. 7).

In some embodiments, in step 306 ridge regression was performed with 10-fold cross validation to determine the optimal lambda. FIG. 8 depicts a plot where the value of lambda for ridge regression is determined in step 306. The dashed line indicates the optimal lambda (where binominal deviance is minimized) (FIG. 8).

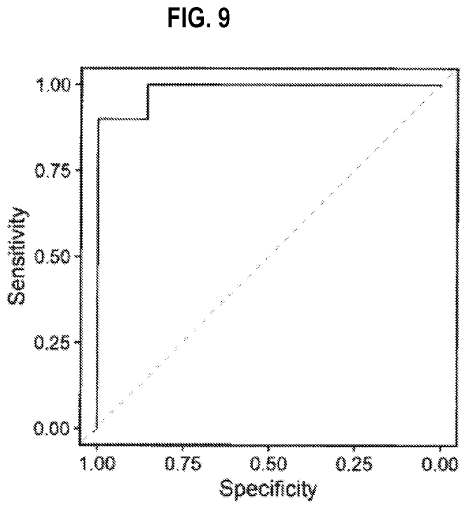

In an example embodiment, the model is determined in step 306 with the optimal lambda retained for the analyses. Using this model, a Receiver Operating Characteristic (ROC) curve was generated (FIG. 9).

In an example embodiment, in step 204 the determined prediction value is compared with a threshold value, in order to assess a risk of the patient having high bone loss (e.g., above 10%). In this example embodiment, the optimum probability threshold for the model was determined to be between 0.430 and 0.548. (This means that if step 204 returns a prediction greater than this value, then the method 200 predicts that the patient belongs in a high-risk category of someone with at least 10% bone loss).

The accuracy of the method 200 for predicting high bone loss is now discussed. In an embodiment, the prediction of high bone loss made in step 204 is now compared with an actual level of bone loss for one or more patients. Sample predictions determined in step 204 can be found in Table 5. The data in Table 5 compares the determined prediction in step 204 of high bone loss with an actual bone loss for each patient. When this threshold is applied to the diagnosis of 17 subjects, there were 2 incorrect predictions. One is a false positive (0% bone loss, but diagnosis of GLB 10 plus) and the other is false negative (12% bone loss, but diagnosis of GLB under 10). The incorrect predictions are highlighted in Table 5 below.

TABLE 5
Sample prediction

| | | | | | | Glenoid | Hill |
|---|---|---|---|---|---|---|---|
| | | | | | | Bone | Sachs |
| Actual | | Predicted | | Age | | Loss | Length |
| Group | Prediction | Group | PID | (years) | Sex | (%) | (mm) |
| GBLUnder10 | 0.42 | GBLUnder10 | ORTH1.1 | 23 | M | 0 | 17.8 |
| GBL10Plus | 0.85 | GBL10Plus | ORTH7 | 21 | M | 18 | 21 |
| GBL10Plus | 0.93 | GBL10Plus | ORTH9 | 20 | M | 22 | 14 |
| GBL10Plus | 0.94 | GBL10Plus | ORTH10 | 22 | M | 13 | 21 |
| GBL10Plus | 0.95 | GBL10Plus | ORTH19 | 27 | M | 17 | 26 |
| GBLUnder10 | 0.66 | GBL10Plus | ORTH20 | 40 | M | 0 | 17 |
| GBL10Plus | 0.44 | GBLUnder10 | ORTH21 | 24 | M | 12 | 20.4 |
| GBLUnder10 | 0.39 | GBLUnder10 | ORTH24 | 21 | M | 8 | 14 |
| GBL10Plus | 0.89 | GBL10Plus | ORTH40 | 24 | M | 15 | 15 |
| GBLUnder10 | 0.32 | GBLUnder10 | ORTH45 | 21 | M | 0 | 18 |
| GBL10Plus | 0.88 | GBL10Plus | ORTH48 | 20 | M | 10 | 23 |
| GBLUnder10 | 0.04 | GBLUnder10 | ORTH50 | 41 | F | 0 | 15 |
| GBL10Plus | 0.70 | GBL10Plus | ORTH51 | 29 | M | 22 | 18.5 |
| GBL10Plus | 0.76 | GBL10Plus | ORTH52 | 24 | M | 25 | 20 |
| GBLUnder10 | 0.05 | GBLUnder10 | ORTH53 | 39 | M | 0 | 20 |
| GBLUnder10 | 0.00 | GBLUnder10 | ORTH54 | 21 | M | 8 | 14 |
| GBL10Plus | 0.78 | GBL10Plus | ORTH35 | 25 | M | 10 | 27 |

The model's metrics are reproduced in Table 6.

TABLE 6
Model Metrics

| | | conf. | conf. | | conf. |
|---|---|---|---|---|---|
| term | estimate | low | high | p.value | hi |
| accuracy | 0.88 | 0.64 | 0.99 | 0.01 | |
| kappa | 0.76 | | | | |
| mcnemar | | | | 1.00 | |
| sensitivity | 0.90 | | | | |
| specificity | 0.86 | | | | |
| pos_pred_value | 0.90 | | | | |
| neg_pred_value | 0.86 | | | | |
| precision | 0.90 | | | | |
| recall | 0.90 | | | | |
| f1 | 0.90 | | | | |
| prevalence | 0.59 | | | | |
| detection_rate | 0.53 | | | | |
| detection_prevalence | 0.59 | | | | |
| balanced_accuracy | 0.88 | | | | |
| auc | 0.99 | 0.95 | | | 1.00 |

where:

$$Accuracy = ( true positives + true negatives ) / total patient number$$

$$Sensitivity = true positives / ( true positives + false negatives )$$

$$Specificity = true negatives / ( false positives + true negatives )$$

$$Positive predictive value = true positives / ( true positives + false positives )$$

$$Negative predictive value = true negatives / ( false negatives + true negatives )$$

$$Precision = true positives / ( true positives + flase positives ) .$$

$$Precision describes how good a model is at predicting the positive class .$$

$$Recall = true positives / ( true positives + false negatives ) .$$

$$F 1 score = 2 \times ( precision \times recall ) / ( precision + recall ) .$$

$$A higher F 1 score denotes a better quality classifier .$$

Sensitivity measures how good the test is at correctly identifying ‘diseased’ individuals and Specificity measures how good the test is at correctly identifying ‘non-diseased’ individuals. Ideally good and accurate tests should have both sensitivity and specificity close to 1 (or 100%). In this model, they are 0.90 and 0.86, respectively, indicating the model is a good test, which is also proved by accuracy of 0.88.

The Area under the ROC (AUC) Curve, which measures the entire two-dimensional area underneath the entire ROC curve from (0,0) to (1,1), indicates how good the diagnostic test is over all cut points. AUC provides an aggregate measure of performance across all possible classification thresholds. In a perfect diagnostic test, the AUC is 1 (1×1=1), and usually values over 0.7 indicates a good diagnostic test and values over 0.8 indicates a strong diagnostic test. In this model, the AUC is 0.99, indicating the model is a very strong diagnostic test.

Gene ontology (GO) analysis was performed. No cellular components (CC), metabolic functions (MF), or biological processes (BP) were identified as statistically significant.

Normalized gene expression was plotted as percentage expression relative to the average of the <10% group (FIGS. 10A through 10E). The gene expression of 4 genes—IFI44, IFIT1, IFIT4, and OAS2—decreased in GBL patients of greater than or equal to 10% bone loss, and the gene expression of PRKCB increased in the GBL patients of greater than or equal to 10% bone loss.

In summary, a peripheral blood biomarker was developed, which reliably identifies those recurrent anterior shoulder instability patients with significant glenoid bone loss greater than or equal to 10%. This is done by evaluating the differential gene expression in blood samples of anterior shoulder instability patients with and without significant glenoid bone loss. Five differentially expressed genes between the 2 groups were identified, and then a predictive model was developed, which discriminates between low and high glenoid bone loss. The receiver operating characteristic curve (ROC) for the model has an area under the curve (AUC) of 0.95 to 0.99.

Based on these results, a method 300 to develop a classifier model for the diagnosis of glenoid bone loss and a test kit to determine the levels of glenoid bone loss biomarker gene expression using a real time quantitative polymerase chain reaction (rt-qPCR) or multiplex-qPCR is disclosed.

In some embodiments, the method comprises steps of:
collecting specimens from patients and non-patients;
extracting RNA from the specimens;
performing transcriptome gene expression analysis;
quantifying gene expression levels;
identifying genes expressed at different levels between patients and non-patients;
performing differential gene expression analysis with the fold-increase or decrease numbers of the identified genes;
establishing a ridge regression model with the results of the differential gene expression analysis of the identified genes;
determining the threshold value;
and testing the model by calculating metrics including sensitivity, specificity, and AUC.

In an embodiment, in method 300 the specimen or sample 103 is blood, lymphatic fluid, cells, tissues, or organs, the step of transcriptome gene expression analysis (e.g. performed by the device 102) can be microarray, RNA-sequencing or nCounter gene expression analysis, the step 306 of fitting data includes differential gene expression analysis comprises steps of normalization and Principal Component Analysis (PCA), and the identified genes are IFIT1, IFIT3, IFI44, PRKCB, and OAS2.

In some embodiments, a kit for rt-qPCR or multiplex qPCR comprises;
oligonucleotide primers complementary to the target sequences of IFIT1 and probes comprising oligonucleotide complementary to another target sequence in the 3′ direction of the primers and labeled with detachable fluorophore for mRNA encoding IFIT1;
oligonucleotide primers complementary to the target sequences of IFIT3 and probes comprising oligonucleotide complementary to another target sequence in the 3′ direction of the primers and labeled with detachable fluorophore for mRNA encoding IFIT3;
oligonucleotide primers complementary to the target sequences of IFI44 and probes comprising oligonucleotide complementary to another target sequence in the 3′ direction of the primers and labeled with detachable fluorophore for mRNA encoding IFI44;
oligonucleotide primers complementary to the target sequences of PRKCB and probes comprising oligonucleotide complementary to another target sequence in the 3′ direction of the primers and labeled with detachable fluorophore for mRNA encoding PRKCB;
oligonucleotide primers complementary to the target sequences of OAS2 and probes comprising oligonucleotide complementary to another target sequence in the 3′ direction of the primers and labeled with detachable fluorophore for mRNA encoding OAS2; and
oligonucleotide primers complementary to the target sequences of a housekeeping gene and probes comprising oligonucleotide complementary to another target sequence in the 3′ direction of the primers and labeled with detachable fluorophore for mRNA encoding housekeeping gene, wherein at least one housekeeping gene is selected from CLTC, GAPDH, GUSB, HPRT1, PGK1, or TUBB;
wherein the fluorophores of individual probes can be the same or different in real time qPCR, and wherein the fluorophores of individual probes are different from each other in multiplex qPCR.

As used herein, the term “Principal Component Analysis (PCA)” refers to a dimensionality reduction technique by finding the greatest amounts of variation in a dataset and assigning it to principal components. The principal component (PC) explaining the greatest amount of variation in the dataset is PC1, while the PC explaining the second greatest amount is PC2, and so on and so forth. In some embodiments, PCA is employed during the method 300, such as in step 306.

In an example embodiment, in the method 300, 17 subjects are presented each having 5 different genes on a 2-dimensional space, 5 principal components (PCs) are calculated using the best fitting lines (e.g. in step 306) passing through the origin (the calculated center of the 17 data points in the 5-dimension where each gene represents each axis, theoretically); best fitting line is PC1, 2nd best fitting line PC2, 3rd best fitting line PC3, 4th best fitting line PC4 and 5th best fitting line PC5, and all fitting lines are vertical to each other. Then the ‘loading score’ of each gene is determined for each PC (e.g., vector part contribution of each gene for PC1 line slope in %; unit vector is called eigenvector).

Then, in this example embodiment, in step 306 the eigenvalue is obtained using the Sum of Squared Distances (SS(distances)). The distance (d) is the distance from the origin to the point vertically projected on the PC line from a data point (There will be total 17 ds for 17 data points on each PC line), and the Sum of Squared Distances (SS(distances)) is calculated; for example, SS(distances) for PC1=d₁²+d₂²+d₁₇², and there will be 5 SS(distances)s. Then, eigenvalue or coefficient value for each PC is calculated, which is variation for each PC. For example, eigenvalue for PC1 is SS(distance) divided by (n−1). In such a way, 5 variations for 5 PCs are calculated. Then all 5 variation values are combined, and each variation of PC1, PC2, PC3, PC4, or PC5 is divided by the sum of all 5 variation values, and percentage number for each PC is presented as a screen plot (FIGS. 6A and 6B). Generally, PC1 and PC2 explain the largest amounts of variation in data, and thus are focused on and plotted against each other for 2-dimensional plot. Then, the plot is rotated to make the PC1 line horizontal (new x axis) and PC2 line vertical (new y axis), and the coordinates of the 17 data points are determined using the projected points of the 17 data points on the PC1 and PC2 lines (x, y) (FIG. 7).

As used herein, in an example embodiment, such as in step 306, the term “cross validation” refers to a method to establish a model and to test the model in a data set by dividing the data set with an arbitrary number and using each part for training and testing. For example, 10-fold cross validation means that the whole data set is divided randomly into 10 parts. Nine of those parts are used for training (determining good values for the slope and the y axis intercept in a regression model) and reserve one tenth for testing. This procedure is repeated for 10 times, each time reserving a different tenth for testing.

As used herein, in an example embodiment, such as in regard to step 306, the term “ridge regression” refers to the regularized form of linear regression, which is a line equation of Y=mX+C. In mathematical terms, Y is the dependent value; X is the independent value; m is the slope of the line and a value of a coefficient for the value of the independent variable X; and c is the constant value. However, in machine learning or the statistical terms, Y is the predicted value; X is feature value; m is coefficients or weights; and c is the bias value. In an example embodiment, where 5 different biomarkers are used to determine the prediction Y, the above equation becomes:

Y=m₁X₁+m₂X₂+m₃X₃+m₄X₄+m₅X₅+C, where X₁-X₅ are the values of the different biomarker levels and m₁-m₅ are the values of respective coefficients for each biomarker. The values of the respective coefficients are determined using the method 300 of FIG. 3 when determining a classifier model. Then the above equation can be used in the method of FIG. 2 to determine a prediction (Y) based on the input data (values of X₁-X₅ measured from the patient sample) and the coefficient values (m₁-m₅).

In an example embodiment, such as in regard to step 306, linear regression uses the Least Squares regression line to model the relationship between x and y. In linear regression, a residual means the difference or vertical distance (y-y̌) between the actual value (y) and the value predicted by the model (y̌) for any given point, and the Least Squares regression line is the line that minimizes the sum of the residuals squared (overfit).

Sometimes, in the example embodiment of step 306, when applying the Least Squares regression line obtained using training data to testing data, the sum of the squared residuals is large, meaning the line has high variance. Ridge regression is a new line that doesn't fit the training data, and by introducing a small amount of bias, reduces variances. With a slightly worse fit, ridge regression can provide better long-term predictions. Thus, when ridge regression determines less fitting values for the parameters (slope and y-axis intercept), ‘penalty’ (λ×slope²) is given, and λ decides the severity of the ‘penalty’.

In the example embodiment, such as in regard to step 306, using ridge regression, the equation is

Y=mX+C, which is obtained by minimizing the quantity (sum of the squared residuals+(λ×slope²)). Thus, the higher the value of lambda (λ), the lower the value of the slope m used in determining the equation.
Lambda (λ) can be any value from 0 to positive infinity and determined arbitrarily through 10-fold cross validation to determine the λ value resulting in the lowest variance. When λ is 0, there is no penalty, and the larger λ is, the bigger is the penalty. The ridge regression penalty resulted in a reduced slope. So, the larger λ is, the prediction for y is less sensitive to change in x. Without the small amount of bias that the penalty creates, the Least Square regression line may have a large amount of variance, and when the slope is steep, y is very sensitive to relatively small change in x. Ridge regression is an approach to regain the regression model's stability.

### Examples

The details disclosed in the Examples section are merely example embodiments of the present invention and thus do not limit the scope of the disclosed embodiments.

### Example 1: Methods and Materials

### Experimental Procedures

### Study Design and Patient Characteristics

This study is a prospective evaluation of all patients undergoing surgery for symptomatic recurrent anterior shoulder instability at a single academic institution. Study approval was obtained from our Institutional Review Board (IRB), Protocol #222038, and all subjects provided written informed consent before biologic specimen collection. All patients, age 18 to 45 years old undergoing arthroscopic and open shoulder surgery for recurrent anterior shoulder instability were included. Patients with collagen disorders, posterior or multidirectional shoulder instability, functional shoulder instability (FSI), adhesive capsulitis, or diagnoses other than symptomatic unidirectional anterior shoulder instability were excluded. Seventeen patients were prospectively enrolled. Patients were indicated for arthroscopic or open anterior shoulder surgical stabilization if they had history, physical examination, and advanced imaging (Magnetic Resonance Imaging (MRI)/Magnetic Resonance Arthrogram (MRA) or Computed Tomography (CT) scan) consistent with an anterior inferior glenoid labral tear and symptomatic anterior shoulder instability. The following procedures were performed: (6) Arthroscopic Bankart repair, (6) Arthroscopic Bankart repair with Hill Sachs Remplissage, and (5) open Latarjet.

### Data Collection

All patients underwent a preoperative standard institutional MRI and/or CT scan with 3-dimensional (3D) reconstructions. The glenoid defect area was calculated according to validated methods (Bakshi et al, AJSM 2018) on standardized en face views of a CT scan with 3D reconstruction. For patients without a CT scan with 3D reconstruction, the glenoid defect area was measured on MRI/MRA sagittal oblique sequence. Significant glenoid bone loss was defined as greater than or equal to 10%. Therefore, in step 304 two groups of patients were identified: GBL<10% and GBL 10%. The Hill Sachs length was measured on the axial MRI sequence with a line from the articular insertion of the rotator cuff to the medial margin of the Hill Sachs lesion (Shaha et al, JBJS 2016).

### Patient Blood and Tissue Specimen Collection

Arthroscopic shoulder stabilization procedures were performed in the lateral decubitus (N=11) and beach chair (N=1) positions. All open Latarjet procedures were performed in the beach chair position. After an examination under anesthesia, patients underwent sterile preparation and draping. For arthroscopic shoulder stabilization cases, the arthroscope was first inserted through a standard posterior viewing portal, and a rotator interval portal was established under direct visualization. Loose anterior inferior capsulolabral tissue which was going to be debrided as part of the standard of care for that procedure was collected. For open Latarjet procedures, the resected anterior inferior capsulolabral tissue was sent as a specimen, as the authors do not perform a capsulolabral repair as part of the open Latarjet procedure. The tissue specimens were sectioned and placed into aliquots and frozen at −80 degrees Celsius. Following tissue specimen collection, 3 ml of peripheral blood was collected and placed in a DNA/RNA Shield Blood Collection tube (Zymo Research Corporation, Catalog #R1150, Lot #PNG430299). It was gently inverted 10 times, placed on ice, and transported within 15 minutes to the hospital's research laboratory where it was aliquoted into multiple 1000 ul cryovials and stored at −80 degrees Celsius.

### Total RNA Isolation

RNA from snap frozen tissues was extracted by first pulverizing tissue in Bio Pulverizer devices super-cooled with liquid nitrogen in the presence of Trizol. Next, the pulverized tissues were funneled into centrifuge tubes, allowed to thaw, and then homogenized with Pro200 for one minute. This mixture was incubated at room temperature for 45 minutes and inverted every 15 minutes. Following this incubation, chloroform was added and the tube shaken. After a final 15 minute incubation, the tubes were centrifuged and extraction performed using the RNAeasy Tissue Lipid RNA kit per the manufacturer's instructions. RNA was extracted from blood preserved in DNA/RNA Shield using the ZymoResearch RNA Whole Blood kit. Briefly, 400 μl thawed blood was processed per the manufacturer's instructions, which included DNase treatment. Elution of RNA was done with 33 μl of 1 mM Tris, pH 8. RNA quality and quantity were assessed using the NanoDrop One, Qubit high sensitivity, and RNA Tapestation. Aliquots of RNA were placed in Lo-Bind tubes and frozen −80 degree Celsius until use.

Real-Time Quantitative Polymerase Chain Reaction (qPCR) Sample Processing

To prepare complimentary DNA (cDNA), total RNAs were reverse transcribed using the SuperScript VILO cDNA synthesis kit (Invitrogen), in a 96 well thin-walled PCR plate on the QIAamplifier (Qiagen). Real time relative qPCR was performed using Taqman predesigned assays. The default thermocycling program was selected, using Fast Advanced Master Mix (Applied Biosystems), and samples were run in triplicate or quadruplicate. A ‘No template control’ was included on each PCR plate.

nCounter Sample Processing

Gene expression was assayed on an nCounter Sprint using the Human Inflammation Panel customized to include additional probes relevant to the question of interest, such as cartilage oligomeric matrix protein (COMP). For each nCounter run, twelve RNA samples were normalized to 10 ng/μl. Five microliters of each were added to 10 μl of Reporter CodeSet and Reporter Plus Master Mix. The Capture CodeSet and Capture Plus Master Mix were prepared and 3 μl were added to the samples. These were hybridized for 16 hours at 65° C. with a heated lid at 70° C. The Sprint cartridge was loaded with the 12 samples and run on the Sprint Profiler.

### Statistical Analysis

Transcript count data generated on an nCounter were exported to CSV files and imported into R using the NanoStringDiff package (Wang H, Nucleic Acids Res 2016). The same package was used to perform differential expression analysis to determine whether there were differences in gene expression between the <10% glenoid bone loss (GBL) and 210% GBL groups. Wilcoxon rank sum tests were performed on the differentially expressed genes to identify a subset of genes with high confidence of being differentially expressed. The raw gene expression data were normalized by dividing gene expression by the geometric average of the expression of the housekeeping genes. The subset of normalized and differentially expressed genes were subjected to Principal Components Analysis (PCA). Ridge regression was performed to predict GBL group based on gene expression levels. The genes included in this model were the differentially expressed genes identified in the above analysis (IFIT1, OAS2, PRKCB, IFIT3, IFI44) after normalization against housekeeping genes (CLTC, GAPDH, GUSB, HPRT1, PGK1, TUBB) as described above. Normalized qPCR results were analyzed by t-test. Statistical analyses were performed in R version 4.0.2.

### Example 2: Patients Selection

Seventeen patients (16 males, 1 female) undergoing surgery for recurrent anterior shoulder instability were prospectively enrolled with a mean age of 26 years (range, 20-41). Seven patients had <10% GBL with a mean GBL of 2.3% (range, 0-8) and 10 patients had 10% GBL with a mean GBL of 16.4% (range, 10-25).

### Example 3: Tissue Specimens

No genes in the shoulder anterior capsular specimens were differentially expressed (Table 1). Nine genes were identified as significantly differentially expressed in the peripheral blood (Table 2). Five of these IFIT1, IFIT3, IFI44, PRKCB, OAS2 (P values of 1×10-5, 1×10-4, 1×10-4, 1×10-4, and 6×10-4) were confirmed using non-parametric tests (Table 3).

### Example 4: Transcriptomic Classifier to Predict Severity of GBL

A predictive model was developed in the method 300 (ridge regression) using the 5 differentially expressed genes in the blood to accurately predict the severity of GBL (<10% GBL and ≥10% GBL). The predictive model had an accuracy of 88% (95% Cl of 64% to 99%), a sensitivity of 0.90, a specificity of 0.86, and an area under the receiver operating characteristic (AUROC) curve of 0.99 (95% Cl of 0.95 to 1.00).

### Example 5: Discussion of Examples 1-7

The primary findings of this study are that there are significant gene expression differences in the peripheral blood of anterior shoulder instability patients with and without significant (10%) GBL. Five inflammatory genes were differentially expressed between the 2 groups: IFIT1, IFIT3, IFI44, PRKCB, OAS2 (P values of 1×10-5, 1×10-4, 1×10-4, 1×10-4, and 6×10-4). A ridge regression model was used to develop a peripheral blood transcriptomic classifier to accurately predict the severity of GBL in patients undergoing surgery for recurrent anterior shoulder instability. The predictive model had an accuracy of 88% (95% Cl of 64% to 99%), a sensitivity of 0.90, a specificity of 0.86, and an area under the receiver operating characteristic (AUROC) curve of 0.99 (95% Cl of 95% to 100%). This novel transcriptomic data may provide a relevant biomarker to track injury severity and progression in patients with recurrent anterior shoulder instability.

Few studies have examined the role of biomarkers in the diagnosis and management of patients with shoulder instability. Yu et al. conducted a study of 33 patients undergoing surgical shoulder stabilization to determine whether preoperative biomarkers of cartilage turnover and inflammation were associated with specific shoulder lesions in shoulder instability (Yu et al, Translational Sports Medicine). Protein biomarkers, cartilage oligomeric matrix protein (COMP), C-reactive protein (HS-CRP), interleukin-8 (IL-8), and macrophage inflammatory protein-1β (MIP-1b) were collected at the time of surgery. Patients with Hill Sachs lesions had a 31% increase in COMP plasma levels (p=0.046). No other significant differences were observed for any of the protein biomarkers including Hill-Sachs lesions, capsular injuries, bony Bankart fractures, and SLAP lesions.

Belangero et al (Revbrasortop, 2014) sampled the injured anterior inferior glenohumeral capsule and compared this to the unaffected anterior superior capsular region in 18 patients with traumatic anterior shoulder instability. The authors found reduced expression of one of the collagen genes (COL5A1). In a subsequent study the same group evaluated tissue samples of recurrent anterior shoulder instability patients compared to controls, and found that recurrent shoulder dislocation presented upregulation of TGFβR1 in the antero-inferior capsule, which is a gene involved in the regulation of collagen cross-linking (Belangero et al, J Orthop Res. 2016). Although this data is interesting and provides improved understanding of the pathobiology, it is not helpful to clinicians in their management of patients. The following collagen genes were included (COL1A1, COL1A2, COL3A1, COL4A2, COL5A1, COL5A2, COL6A3) in a customized 277-gene expression panel (nCounter) and did not find any differential expression in the capsular tissue or blood between the low and high GBL groups.

Recently, Aleem et al examined the gene expression in glenoid cartilage tissue samples among patients with acute instability (<3 dislocations), chronic instability (≥3 dislocations), and glenohumeral osteoarthritis. They found that glenoid articular cartilage in the osteoarthritis group displayed higher expression of CCL3, CHST11, GPR22, PRKAR2B, and PTGS2 than cartilage in the group with acute or chronic shoulder instability. CCL3 was identified as a gene of interest. In the analysis CCL3 was not significantly different in the anterior capsular tissue specimens (p value=0.018, q value=0.66). It was significantly different in the blood (p value=0.00013, q value=0.008); however, with non-parametric confirmation testing, the result was not significant (p value=0.07). In the blood it demonstrated that the CCL3 gene was downregulated in the high bone loss group (log FC=−2.3).

IFIT1, IFIT3 and IFI44 were all significantly differentially expressed genes between patients with and without significant GBL. All of these genes were overexpressed in patients with less than 10% GBL. The mechanism for these findings are unclear. Prior literature has found that IFIT1 could regulate Wnt/p-catenin signaling (Li TH, Cell Oncol (Dordr). 2021). This pathway comprises a family of proteins that play critical roles in tissue homeostasis. Specifically, the canonical Wnt signaling pathway affects the proliferation and differentiation of mesenchymal stem cells, osteoblast progenitor cells in addition to osteoclast function. This pathway is critical to maintenance of bone homeostasis (Liu et al, Sig Transduct Target Ther 2022).

PRKCB gene was significantly overexpressed in the patients with greater than or equal to 10% GBL. This gene has been well studied and interestingly it is also significantly overexpressed in Ewing's Sarcoma. Surdez et al found that the transcriptional activation of PRKCB was directly regulated by the chimeric fusion oncogene EWSR1-FL11 which drives the growth of the malignant bone tumor, Ewing Sarcoma (Surdez D et al, Cancer research 2012). Additionally, the authors discovered that PRKCB loss induced apoptosis in vitro and prevented tumor growth in vivo. These findings provided a proof of concept for potential targeting of PRKCB gene as a therapeutic for Ewing Sarcoma treatment. The mechanism of PRKCB overexpression in patients with increasing glenoid bone loss is unclear; however, perhaps there is a mechanism of high bone turnover in GBL that is similar to that seen in Ewing's Sarcoma (Zota V et al, Fetal Pediatr Pathol. 2022).

Prior musculoskeletal studies have attempted to identify diagnostic and prognostic biomarkers for use in clinical practice and have been unsuccessful. Further, a number of studies have identified tissue-level significant differences in gene expression; however, this tissue transcriptomic data is less useful in the clinical setting. This novel blood transcriptomic data may provide a potential biomarker to track injury severity and progression in patients with recurrent anterior shoulder instability.

Controversy still exists regarding the optimal treatment for first-time anterior glenohumeral dislocations. Many patients elect for nonoperative treatment due to a number of reasons including in-season athletics, the desire to avoid a surgical intervention, or improvement in function with no persistent apprehension. This novel serum biomarker (transcriptomic classifier) could be useful in this clinical scenario as it could indicate when the injury severity has progressed to a near significant amount of injury that would necessitate surgery. Further, the other clinical scenario where this biomarker may be helpful is in resource-limited environments without ready access to advanced imaging modalities. For patients who elect for nonoperative management after an initial work up with CT scan and MRI/MRA, this biomarker may obviate the need for repeat advanced imaging which imparts radiation to the patient (CT) and is costly (MRI/MRA). Finally, this transcriptomic data has the potential for monitoring of the success of arthroscopic anterior shoulder stabilization; however, further larger prospective investigations are needed to validate the findings in postoperative patients with longitudinal studies.

Limitations of the study include the small sample size, and future larger studies are needed to confirm these findings.

Strengths of the study include the prospective enrollment of patients with collection of tissue and blood biologic specimens. Additionally, another strength is the rigorous scientific method followed with confirmation of RNA quality with high RNA integrity numbers (RINs), and qPCR to verify gene expression findings. Additionally, another strength is the development of a novel accurate statistical model to determine a transcriptomic classifier for the reliable delineation of the severity of GBL.

### Hardware Overview

FIG. 11 is a block diagram that illustrates a computer system 1100 upon which an embodiment of the invention may be implemented. Computer system 1100 includes a communication mechanism such as a bus 1110 for passing information between other internal and external components of the computer system 1100. Information is represented as physical signals of a measurable phenomenon, typically electric voltages, but including, in other embodiments, such phenomena as magnetic, electromagnetic, pressure, chemical, molecular atomic and quantum interactions. For example, north and south magnetic fields, or a zero and non-zero electric voltage, represent two states (0, 1) of a binary digit (bit). Other phenomena can represent digits of a higher base. A superposition of multiple simultaneous quantum states before measurement represents a quantum bit (qubit). A sequence of one or more digits constitutes digital data that is used to represent a number or code for a character. In some embodiments, information called analog data is represented by a near continuum of measurable values within a particular range. Computer system 1100, or a portion thereof, constitutes a means for performing one or more steps of one or more methods described herein.

A sequence of binary digits constitutes digital data that is used to represent a number or code for a character. A bus 1110 includes many parallel conductors of information so that information is transferred quickly among devices coupled to the bus 1110. One or more processors 1102 for processing information are coupled with the bus 1110. A processor 1102 performs a set of operations on information. The set of operations include bringing information in from the bus 1110 and placing information on the bus 1110. The set of operations also typically include comparing two or more units of information, shifting positions of units of information, and combining two or more units of information, such as by addition or multiplication. A sequence of operations to be executed by the processor 1102 constitutes computer instructions.

Computer system 1100 also includes a memory 1104 coupled to bus 1110. The memory 1104, such as a random access memory (RAM) or other dynamic storage device, stores information including computer instructions. Dynamic memory allows information stored therein to be changed by the computer system 1100. RAM allows a unit of information stored at a location called a memory address to be stored and retrieved independently of information at neighboring addresses. The memory 1104 is also used by the processor 1102 to store temporary values during execution of computer instructions. The computer system 1100 also includes a read only memory (ROM) 1106 or other static storage device coupled to the bus 1110 for storing static information, including instructions, that is not changed by the computer system 1100. Also coupled to bus 1110 is a non-volatile (persistent) storage device 1108, such as a magnetic disk or optical disk, for storing information, including instructions, that persists even when the computer system 1100 is turned off or otherwise loses power.

Information, including instructions, is provided to the bus 1110 for use by the processor from an external input device 1112, such as a keyboard containing alphanumeric keys operated by a human user, or a sensor. A sensor detects conditions in its vicinity and transforms those detections into signals compatible with the signals used to represent information in computer system 1100. Other external devices coupled to bus 1110, used primarily for interacting with humans, include a display device 1114, such as a cathode ray tube (CRT) or a liquid crystal display (LCD), for presenting images, and a pointing device 1116, such as a mouse or a trackball or cursor direction keys, for controlling a position of a small cursor image presented on the display 1114 and issuing commands associated with graphical elements presented on the display 1114.

In the illustrated embodiment, special purpose hardware, such as an application specific integrated circuit (IC) 1120, is coupled to bus 1110. The special purpose hardware is configured to perform operations not performed by processor 1102 quickly enough for special purposes. Examples of application specific ICs include graphics accelerator cards for generating images for display 1114, cryptographic boards for encrypting and decrypting messages sent over a network, speech recognition, and interfaces to special external devices, such as robotic arms and medical scanning equipment that repeatedly perform some complex sequence of operations that are more efficiently implemented in hardware.

Computer system 1100 also includes one or more instances of a communications interface 1170 coupled to bus 1110. Communication interface 1170 provides a two-way communication coupling to a variety of external devices that operate with their own processors, such as printers, scanners and external disks. In general, the coupling is with a network link 1178 that is connected to a local network 1180 to which a variety of external devices with their own processors are connected. For example, communication interface 1170 may be a parallel port or a serial port or a universal serial bus (USB) port on a personal computer. In some embodiments, communications interface 1170 is an integrated services digital network (ISDN) card or a digital subscriber line (DSL) card or a telephone modem that provides an information communication connection to a corresponding type of telephone line. In some embodiments, a communication interface 1170 is a cable modem that converts signals on bus 1110 into signals for a communication connection over a coaxial cable or into optical signals for a communication connection over a fiber optic cable. As another example, communications interface 1170 may be a local area network (LAN) card to provide a data communication connection to a compatible LAN, such as Ethernet.

Wireless links may also be implemented. Carrier waves, such as acoustic waves and electromagnetic waves, including radio, optical and infrared waves travel through space without wires or cables. Signals include man-made variations in amplitude, frequency, phase, polarization or other physical properties of carrier waves. For wireless links, the communications interface 1170 sends and receives electrical, acoustic or electromagnetic signals, including infrared and optical signals, that carry information streams, such as digital data.

The term computer-readable medium is used herein to refer to any medium that participates in providing information to processor 1102, including instructions for execution. Such a medium may take many forms, including, but not limited to, non-volatile media, volatile media and transmission media. Non-volatile media include, for example, optical or magnetic disks, such as storage device 1108. Volatile media include, for example, dynamic memory 1104. Transmission media include, for example, coaxial cables, copper wire, fiber optic cables, and waves that travel through space without wires or cables, such as acoustic waves and electromagnetic waves, including radio, optical and infrared waves. The term computer-readable storage medium is used herein to refer to any medium that participates in providing information to processor 1102, except for transmission media.

Common forms of computer-readable media include, for example, a floppy disk, a flexible disk, a hard disk, a magnetic tape, or any other magnetic medium, a compact disk ROM (CD-ROM), a digital video disk (DVD) or any other optical medium, punch cards, paper tape, or any other physical medium with patterns of holes, a RAM, a programmable ROM (PROM), an erasable PROM (EPROM), a FLASH-EPROM, or any other memory chip or cartridge, a carrier wave, or any other medium from which a computer can read. The term non-transitory computer-readable storage medium is used herein to refer to any medium that participates in providing information to processor 1102, except for carrier waves and other signals.

Logic encoded in one or more tangible media includes one or both of processor instructions on a computer-readable storage media and special purpose hardware, such as ASIC *1120.

Network link 1178 typically provides information communication through one or more networks to other devices that use or process the information. For example, network link 1178 may provide a connection through local network 1180 to a host computer 1182 or to equipment 1184 operated by an Internet Service Provider (ISP). ISP equipment 1184 in turn provides data communication services through the public, world-wide packet-switching communication network of networks now commonly referred to as the Internet 1190. A computer called a server 1192 connected to the Internet provides a service in response to information received over the Internet. For example, server 1192 provides information representing video data for presentation at display 1114.

The invention is related to the use of computer system 1100 for implementing the techniques described herein. According to one embodiment of the invention, those techniques are performed by computer system 1100 in response to processor 1102 executing one or more sequences of one or more instructions contained in memory 1104. Such instructions, also called software and program code, may be read into memory 1104 from another computer-readable medium such as storage device 1108. Execution of the sequences of instructions contained in memory 1104 causes processor 1102 to perform the method steps described herein. In alternative embodiments, hardware, such as application specific integrated circuit 1120, may be used in place of or in combination with software to implement the invention. Thus, embodiments of the invention are not limited to any specific combination of hardware and software.

The signals transmitted over network link 1178 and other networks through communications interface 1170, carry information to and from computer system 1100. Computer system 1100 can send and receive information, including program code, through the networks 1180, 1190 among others, through network link 1178 and communications interface 1170. In an example using the Internet 1190, a server 1192 transmits program code for a particular application, requested by a message sent from computer 1100, through Internet 1190, ISP equipment 1184, local network 1180 and communications interface 1170. The received code may be executed by processor 1102 as it is received, or may be stored in storage device 1108 or other non-volatile storage for later execution, or both. In this manner, computer system 1100 may obtain application program code in the form of a signal on a carrier wave.

Various forms of computer readable media may be involved in carrying one or more sequence of instructions or data or both to processor 1102 for execution. For example, instructions and data may initially be carried on a magnetic disk of a remote computer such as host 1182. The remote computer loads the instructions and data into its dynamic memory and sends the instructions and data over a telephone line using a modem. A modem local to the computer system 1100 receives the instructions and data on a telephone line and uses an infra-red transmitter to convert the instructions and data to a signal on an infra-red a carrier wave serving as the network link 1178. An infrared detector serving as communications interface 1170 receives the instructions and data carried in the infrared signal and places information representing the instructions and data onto bus 1110. Bus 1110 carries the information to memory 1104 from which processor 1102 retrieves and executes the instructions using some of the data sent with the instructions. The instructions and data received in memory 1104 may optionally be stored on storage device 1108, either before or after execution by the processor 1102.

FIG. 12 illustrates a chip set 1200 upon which an embodiment of the invention may be implemented. Chip set 1200 is programmed to perform one or more steps of a method described herein and includes, for instance, the processor and memory components described with respect to FIG. *11 incorporated in one or more physical packages (e.g., chips). By way of example, a physical package includes an arrangement of one or more materials, components, and/or wires on a structural assembly (e.g., a baseboard) to provide one or more characteristics such as physical strength, conservation of size, and/or limitation of electrical interaction. It is contemplated that in certain embodiments the chip set can be implemented in a single chip. Chip set 1200, or a portion thereof, constitutes a means for performing one or more steps of a method described herein.

In one embodiment, the chip set 1200 includes a communication mechanism such as a bus 1201 for passing information among the components of the chip set 1200. A processor 1203 has connectivity to the bus 1201 to execute instructions and process information stored in, for example, a memory 1205. The processor 1203 may include one or more processing cores with each core configured to perform independently. A multi-core processor enables multiprocessing within a single physical package. Examples of a multi-core processor include two, four, eight, or greater numbers of processing cores. Alternatively or in addition, the processor 1203 may include one or more microprocessors configured in tandem via the bus 1201 to enable independent execution of instructions, pipelining, and multithreading. The processor 1203 may also be accompanied with one or more specialized components to perform certain processing functions and tasks such as one or more digital signal processors (DSP) 1207, or one or more application-specific integrated circuits (ASIC) 1209. A DSP 1207 typically is configured to process real-world signals (e.g., sound) in real time independently of the processor 1203. Similarly, an ASIC 1209 can be configured to performed specialized functions not easily performed by a general purposed processor. Other specialized components to aid in performing the inventive functions described herein include one or more field programmable gate arrays (FPGA) (not shown), one or more controllers (not shown), or one or more other special-purpose computer chips.

The processor 1203 and accompanying components have connectivity to the memory 1205 via the bus 1201. The memory 1205 includes both dynamic memory (e.g., RAM, magnetic disk, writable optical disk, etc.) and static memory (e.g., ROM, CD-ROM, etc.) for storing executable instructions that when executed perform one or more steps of a method described herein. The memory 1205 also stores the data associated with or generated by the execution of one or more steps of the methods described herein.

### CONCLUSION

There are significant gene expression differences in the peripheral blood of anterior shoulder instability patients with and without significant (?10%) GBL. This novel transcriptomic data may lead to a relevant biomarker to track glenoid bone loss and injury severity and progression in young patients with recurrent anterior shoulder instability. This may be of clinical utility in monitoring injury progression in patients undergoing nonoperative treatment, in-season athletes, and those in resource-limited environments without access to serial advanced imaging.

### Alternatives, Deviations, and Modifications

In the foregoing specification, the invention has been described with reference to specific embodiments thereof. It will, however, be evident that various modifications and changes may be made thereto without departing from the broader spirit and scope of the invention. The specification and drawings are, accordingly, to be regarded in an illustrative rather than a restrictive sense. Throughout this specification and the claims, unless the context requires otherwise, the word “comprise” and its variations, such as “comprises” and “comprising,” will be understood to imply the inclusion of a stated item, element or step or group of items, elements or steps but not the exclusion of any other item, element or step or group of items, elements or steps. Furthermore, the indefinite article “a” or “an” is meant to indicate one or more of the item, element or step modified by the article.

Notwithstanding that the numerical ranges and parameters setting forth the broad scope are approximations, the numerical values set forth in specific non-limiting examples are reported as precisely as possible. Any numerical value, however, inherently contains certain errors necessarily resulting from the standard deviation found in their respective testing measurements at the time of this writing. Furthermore, unless otherwise clear from the context, a numerical value presented herein has an implied precision given by the least significant digit. Thus, a value 1.1 implies a value from 1.05 to 1.15. The term “about” is used to indicate a broader range centered on the given value, and unless otherwise clear from the context implies a broader range around the least significant digit, such as “about 1.1” implies a range from 1.0 to 1.2. If the least significant digit is unclear, then the term “about” implies a factor of two, e.g., “about X” implies a value in the range from 0.5× to 2X, for example, about 100 implies a value in a range from 50 to 200. Moreover, all ranges disclosed herein are to be understood to encompass any and all sub-ranges subsumed therein. For example, a range of “less than 10” for a positive only parameter can include any and all sub-ranges between (and including) the minimum value of zero and the maximum value of 10, that is, any and all sub-ranges having a minimum value of equal to or greater than zero and a maximum value of equal to or less than 10, e.g., 1 to 4.

### REFERENCES

1. Aleem A W, Rai M F, Cai L, Brophy R H. Gene Expression in Glenoid Articular Cartilage Varies Across Acute Instability, Chronic Instability, and Osteoarthritis. J Bone Joint Surg Am. 2023 Apr. 3. doi: 10.2106/JBJS.22.01124. Epub ahead of print. PMID: 37011069.
2. Arciero R A, Wheeler J H, Ryan J B, McBride J T. Arthroscopic Bankart repair versus nonoperative treatment for acute, initial anterior shoulder dislocations. Am J Sports Med. 1994; 22(5):589-94.https://doi.org/10.1177/036354659402200504.
3. Bakshi N K, Cibulas G A, Sekiya J K, Bedi A. A Clinical Comparison of Linear- and Surface Area-Based Methods of Measuring Glenoid Bone Loss: Response. Am J Sports Med. 2019 March; 47(4):NP29-NP30. doi: 10.1177/0363546519825630. PMID: 30870035.
4. Belangero P S, Leal M F, Figueiredo E A, Cohen C, Pochini Ade C, Smith M C, Andreoli C V, Belangero S I, Ejnisman B, Cohen M. Gene expression analysis in patients with traumatic anterior shoulder instability suggests deregulation of collagen genes. J Orthop Res. 2014 October; 32(10):1311-6. doi: 10.1002/jor.22680. Epub 2014 Jul. 11. PMID: 25042113.
5. Belangero P S, Leal M F, de Castro Pochini A, Andreoli C V, Ejnisman B, Cohen M. Profile of collagen gene expression in the glenohumeral capsule of patients with traumatic anterior instability of the shoulder. Rev Bras Ortop. 2014 Oct. 23; 49(6):642-6. doi: 10.1016/j.rboe.2014.10.008. PMID: 26229875; PMCID: PMC4487492.
6. Belangero P S, Leal M F, Figueiredo E A, Cohen C, Andreoli C V, Smith M C, Pochini Ade C, Ejnisman B, Cohen M. Differential expression of extracellular matrix genes in glenohumeral capsule of shoulder instability patients. Connect Tissue Res. 2016 July; 57(4):290-8. doi: 10.3109/03008207.2016.1173034. Epub 2016 Apr. 19. PMID: 27093129.
7. Belangero P S, Leal M F, Cohen C, Figueiredo E A, Smith M C, Andreoli C V, de Castro Pochini A, Ejnisman B, Cohen M. Expression analysis of genes involved in collagen cross-linking and its regulation in traumatic anterior shoulder instability. J Orthop Res. 2016 March; 34(3):510-7. doi: 10.1002/jor.22984. Epub 2015 Aug. 4. PMID: 26185036.
8. Bottoni C R, Wilckens J H, DeBerardino T M, D′Alleyrand J C, Rooney R C, Harpstrite J K, et al. A prospective, randomized evaluation of arthroscopic stabilization versus nonoperative treatment in patients with acute, traumatic, first-time shoulder dislocations. Am J Sports Med. 2002; 30(4):576-80.
9. DeBerardino T M, Arciero R A, Taylor D C, Uhorchak J M. Prospective evaluation of arthroscopic stabilization of acute, initial anterior shoulder dislocations in young athletes. Two- to five-year follow-up. Am J Sports Med. 2001; 29(5):586-92. https://doi.org/10.1177/03635465010290051101.
10. Dickens J F, Slaven S E, Cameron K L, Pickett A M, Posner M, Campbell S E, Owens B D. Prospective Evaluation of Glenoid Bone Loss After First-time and Recurrent Anterior Glenohumeral Instability Events. Am J Sports Med. 2019 April; 47(5):1082-1089. doi: 10.1177/0363546519831286. PMID: 30943084.
11. Di Giacomo G, Itoi E, Burkhart S S. Evolving concept of bipolar bone loss and the Hill-Sachs lesion: from “engaging/non-engaging” lesion to “on-track/off-track” lesion. Arthroscopy. 2014 January; 30(1):90-8. doi: 10.1016/j.arthro.2013.10.004. PMID: 24384275.
12. Eager J M, Warrender W J, Deusenbery C B, Jamgochian G, Singh A, Abboud J A, Spiller K L. Distinct Gene Expression Profile in Patients With Poor Postoperative Outcomes After Rotator Cuff Repair: A Case-Control Study. Am J Sports Med. 2021 August; 49(10):2760-2770. doi: 10.1177/03635465211023212. Epub 2021 Jul. 20. PMID: 34283947.
13. Eichinger J K, Massimini D F, Kim J, Higgins L D. Biomechanical Evaluation of Glenoid Version and Dislocation Direction on the Influence of Anterior Shoulder Instability and Development of Hill-Sachs Lesions. Am J Sports Med. 2016 November; 44(11):2792-2799. doi: 10.1177/0363546516659281. Epub 2016 Aug. 5. PMID: 27496904.
14. Gordon J A, Farooqi A S, Rabut E, Huffman G R, Schug J, Kelly J D, Dodge G R. Evaluating whole-genome expression differences in idiopathic and diabetic adhesive capsulitis. J Shoulder Elbow Surg. 2022 January; 31(1):e1-e13. doi: 10.1016/j.jse.2021.06.016. Epub 2021 Aug. 2. PMID: 34352401; PMCID: PMC8665043.
15. Leal M F, Belangero P S, Cohen C, Figueiredo E A, Loyola L C, Pochini A C, Smith M C, Andreoli C V, Belangero S I, Ejnisman B, Cohen M. Identification of suitable reference genes for gene expression studies of shoulder instability. PLoS One. 2014 Aug. 14; 9(8):e105002. doi: 10.1371/journal.pone.0105002. PMID: 25122470; PMCID: PMC4133370.
16. Li T H, Zhao B B, Qin C, Wang Y Y, Li Z R, Cao H T, Yang X Y, Zhou X T, Wang W B. IFIT1 modulates the proliferation, migration and invasion of pancreatic cancer cells via Wnt/p-catenin signaling. Cell Oncol (Dordr). 2021 December; 44(6):1425-1437. doi: 10.1007/s13402-021-00651-8. Epub 2021 Nov. 18. PMID: 34791638; PMCID: PMC8648688
17. Liu, J., Xiao, Q., Xiao, J. et al. Wnt/P-catenin signalling: function, biological mechanisms, and therapeutic opportunities. Sig Transduct Target Ther 7, 3 (2022). https://doi.org/10.1038/s41392-021-00762-6
18. Moroder P, Damm P, Wierer G, Bohm E, Minkus M, Plachel F, Mardian S, Scheibel M, Khatamirad M. Challenging the Current Concept of Critical Glenoid Bone Loss in Shoulder Instability: Does the Size Measurement Really Tell It All?Am J Sports Med. 2019 March; 47(3):688-694. doi: 10.1177/0363546518819102. Epub 2019 Jan. 14. PMID: 30640513.
19. Owens B D, Cameron K L, Bokshan S L, et al. Serum Cartilage Biomarkers and Shoulder Instability. Orthopedics. 2017 Jan. 1; 40(1):34-36. doi: 10.3928/01477447-20160926-04. Epub 2016 Sep. 30.
20. Owens B D, Cameron K L, Clifton K B, Svoboda S J, Wolf J M. Association Between Serum Relaxin and Subsequent Shoulder Instability. Orthopedics. 2016 Jul. 1; 39(4):e724-8. doi: 10.3928/01477447-20160421-01. Epub 2016 Apr. 27. PMID: 27111077.
21. Provencher M T, Bell S J, Menzel K A, Mologne T S. Arthroscopic treatment of posterior shoulder instability: results in 33 patients. Am J Sports Med. 2005 October; 33(10):1463-71. doi: 10.1177/0363546505278301. Epub 2005 Aug. 10. PMID: 16093530.
22. Rai M F, Cai L, Tycksen E D, Chamberlain A, Keener J. RNA-Seq analysis reveals sex-dependent transcriptomic profiles of human subacromial bursa stratified by tear etiology. J Orthop Res. 2022 December; 40(12):2713-2727. doi: 10.1002/jor.25316. Epub 2022 Mar. 24. PMID: 35266580.
23. Rai M F, Cai L, Tycksen E D, Keener J, Chamberlain A. RNA-Seq reveals distinct transcriptomic differences in rotator cuff tendon based on tear etiology and patient sex. J Orthop Res. 2022 December; 40(12):2728-2742. doi: 10.1002/jor.25296. Epub 2022 Feb. 16. PMID: 35174534.
24. Shaha J S, Cook J B, Rowles D J, Bottoni C R, Shaha S H, Tokish J M. Clinical Validation of the Glenoid Track Concept in Anterior Glenohumeral Instability. J Bone Joint Surg Am. 2016 Nov. 16; 98(22):1918-1923. doi: 10.2106/JBJS.15.01099. PMID: 27852909.
25. Surdez D, BENETKIEWICZ M, GEOERGER B, et al. Targeting the EWSR1-FL11 Oncogene-Induced Protein Kinase PKC-p Abolishes Ewing Sarcoma Growth. Cancer research (Chicago, III). 2012; 72(17):4494-4503. doi:10.1158/0008-5472.CAN-12-0371.
26. Vaswani R, Gasbarro G, Como C, Golan E, Fourman M, Wilmot A, Borrero C, Vyas D, Lin A. Labral Morphology and Number of Preoperative Dislocations Are Associated With Recurrent Instability After Arthroscopic Bankart Repair. Arthroscopy. 2020 April; 36(4):993-999. doi: 10.1016/j.arthro.2019.11.111. Epub 2019 Nov. 27. PMID: 31785391.
27. Wang H, Horbinski C, Wu H, Liu Y, Sheng S, Liu J, Weiss H, Stromberg A J, Wang C. NanoStringDiff: a novel statistical method for differential expression analysis based on NanoString nCounter data. Nucleic Acids Res. 2016 Nov. 16; 44(20):e151. doi: 10.1093/nar/gkw677. Epub 2016 Jul. 28. PMID: 27471031; PMCID: PMC5175344.
28. Wolfe J A, Elsenbeck M, Nappo K, Christensen D, Waltz R, LeClere L, Dickens J F. Effect of Posterior Glenoid Bone Loss and Retroversion on Arthroscopic Posterior Glenohumeral Stabilization. Am J Sports Med. 2020 September; 48(11):2621-2627. doi: 10.1177/0363546520946101. Epub 2020 Aug. 19. PMID: 32813547.
29. Yu J S, Dare D M, Edon D, Siantro A L, Sarver D C, Rodeo S, Dines J S, Mendias C L. Shoulder Lesions Do Not Increase Inflammatory Biomarkers in Patients Undergoing Surgery for Glenohumeral Instability: An Exploratory Study. Translational Sports Medicine 2022 February; Vol 2022. Doi: 10.1155/2022/4220356
30. Zota V, Siegal G P, Kelly D, Bridge J A, Berglund A, Bui K, Khalil F, R Reed D, Altiok S, Magliocco A, Bui M M. Validation of PRKCB Immunohistochemistry as a Biomarker for the Diagnosis of Ewing Sarcoma. Fetal Pediatr Pathol. 2022 Sep. 5:1-12. doi: 10.1080/15513815.2022.2117579. Epub ahead of print. PMID: 36062956.)

What is claimed is:

1. A method comprising:
a) receiving, at a processor, first data that indicates measured values of a panel of biomarkers in a sample from a patient; and
b) determining, with the processor, second data that indicates a prediction that the patient has bone loss above a threshold value based on the first data and a classifier model trained on values of the panel of biomarkers as input and observed bone loss above a threshold value as output, and wherein the method optionally further comprises (i) transmitting, with the processor, a signal to a display to output third data on the display based on the determined prediction; and/or (ii) measuring, with a device, the values of the panel of biomarkers in the sample.

2. (canceled)

3. The method of claim 1, wherein the classifier model comprises coefficients for the measured value of each biomarker in the panel and wherein step b) further comprises applying, on the processor, the coefficients to the measured values of each biomarker such that the determined prediction is based on the first data and the applying the coefficients to the measured values.

4. (canceled)

5. The method of claim 2, wherein the third data (i) indicates the determined prediction or a risk category of the patient for bone loss based on the determined prediction and/or (ii) outputs a treatment protocol for the patient based on the determined prediction.

6. (canceled)

7. The method of claim 1, wherein the classifier model has a performance of a Receiver Operator Characteristic (ROC) curve with a sensitivity value of at least 0.90 and a specificity value of at least 0.86.

8. The method of claim 1, wherein the first data comprises measured values from a panel of three or more biomarkers or five or more bio markers.

9. (canceled)

10. The method of claim 8, wherein the panel of biomarkers are expression products of genes selected from IFIT1, IFIT3, IFI44, PRKCB, and OAS2.

11. The method of claim 10, wherein the classifier model comprises coefficients for the measured values of each biomarker and wherein step b) further comprises applying, on the processor, the coefficients to the measured values of each biomarker.

12. The method of claim 1, wherein the bone loss is glenoid bone loss,
wherein the threshold value is about 10%, and/or
wherein the sample is a blood sample.

13. (canceled)

14. (canceled)

15. The method of claim 1, wherein the classifier model is determined by:
obtaining, at the processor, preliminary data from a plurality of subjects that indicate the measured values of the panel of biomarkers in a same from each of the plurality of subjects;
assigning, on the processor, a result for each subject based on whether the subject has bone loss above the threshold value; and
fitting, on the processor, the preliminary data to the results for the plurality of subjects; and
determining, on the processor, coefficients for each biomarker in the panel to determine the one or more classifier models for predicting whether a patient has bone loss above the threshold value based on the first data.

16. The method of claim 15, wherein the fitting comprises principal component analysis (PCA) and determining, for each biomarker, a value indicating a level of variance between subjects with bone loss above the threshold value and subjects with bone less than the threshold value, and wherein, optionally, the fitting comprises selecting a first group of biomarkers among the plurality of biomarkers whose value indicting the level of variance is greater than a second group of biomarkers within the plurality of biomarkers.

17. (canceled)

18. The method of claim 17, wherein the fitting comprises ridge regression.

19. (canceled)

20. (canceled)

21. (canceled)

22. (canceled)

23. (canceled)

24. (canceled)

25. (canceled)

26. A test kit to determine the levels of glenoid bone loss biomarker gene expression using a real time quantitative polymerase chain reaction (rt-qPCR) or multiplex-qPCR, comprising;
oligonucleotide primers complementary to the target sequences of IFIT1 and probes comprising oligonucleotide complementary to another target sequence in the 3′ direction of the primers and labeled with detachable fluorophore for mRNA encoding IFIT1;
oligonucleotide primers complementary to the target sequences of IFIT3 and probes comprising oligonucleotide complementary to another target sequence in the 3′ direction of the primers and labeled with detachable fluorophore for mRNA encoding IFIT3;
oligonucleotide primers complementary to the target sequences of IFI44 and probes comprising oligonucleotide complementary to another target sequence in the 3′ direction of the primers and labeled with detachable fluorophore for mRNA encoding IFI44;
oligonucleotide primers complementary to the target sequences of PRKCB and probes comprising oligonucleotide complementary to another target sequence in the 3′ direction of the primers and labeled with detachable fluorophore for mRNA encoding PRKCB;
oligonucleotide primers complementary to the target sequences of OAS2 and probes comprising oligonucleotide complementary to another target sequence in the 3′ direction of the primers and labeled with detachable fluorophore for mRNA encoding OAS2; and
oligonucleotide primers complementary to the target sequences of a housekeeping gene and probes comprising oligonucleotide complementary to another target sequence in the 3′ direction of the primers and labeled with detachable fluorophore for mRNA encoding housekeeping gene, wherein at least one housekeeping gene is selected from CLTC, GAPDH, GUSB, HPRT1, PGK1, or TUBB;
wherein the fluorophores of individual probes can be the same or different in real time qPCR, and wherein the fluorophores of individual probes are different from each other in multiplex qPCR.

27. A method of diagnosing and treating whether a subject has suffering from glenoid bone loss of greater than or equal to 10%, comprising:
Determining gene expression levels of IFIT1, IFIT3, IFI44, PRKCB, and OAS2 and, optionally, a housekeeping gene, in a biological sample of a subject by transcriptome gene expression analysis or by real time qPCR or multiplex qPCR using the kit of claim 26;
determining a prediction probability value of the subject using a classifier model trained on input data of expression levels of IFIT1, IFIT3, IFI44, PRKCB, and/or OAS2 from subjects known to have glenoid bone loss and subjects known to not have glenoid bone loss;
Comparing the prediction probability value with a predetermined threshold value; and
Performing surgery on the subject selected from open Latarjet procedure, augmentation with an iliac crest bone graft, glenoid osteochondral allograft, or distal tibia osteochondral allograft if the prediction value is higher than the threshold value.

28. A method of diagnosing and treating a subject suffering from glenoid bone loss, comprising:
determining whether the subject has glenoid bone loss by measuring an amount of expression products of marker genes in a sample from the subject, wherein the marker genes are selected from IFIT1, IFIT3, IFI44, PRKCB, and/or OAS2; wherein when levels of the expression products comprises a deviation relative to control level, indicates glenoid bone loss in the subject; and
Performing surgery on the subject selected from open Latarjet procedure, augmentation with an iliac crest bone graft, glenoid osteochondral allograft, or distal tibia osteochondral allograft.
